(19) United States
(12) Patent Application Publication
(10) Pub. No.: US 2026/0232605 A1
(43) Pub. Date: Aug. 13, 2026

(54) TRANSDERMAL THERAPEUTIC SYSTEM FOR THE TRANSDERMAL ADMINISTRATION OF HUPERZINE A

(71) Applicant: LTS LOHMANN THERAPIE-SYSTEME AG, Andernach (DE)

(72) Inventors: Florian HAMMES, Andernach (DE); Anja TOMELERI, Neuwied (DE); Tobias KLEUDGEN, Ettringen (DE); Patrick MOHR, Bad Breisig (DE)

(21) Appl. No.: 19/150,981

(22) PCT Filed: Feb. 1, 2024
(86) PCT No.: PCT/EP2024/052465

§ 371 Date: Jul. 25, 2025

(30) Foreign Application Priority Data
Feb. 1, 2023 (EP) 23154532.8

(51) Int. Cl. A61K 9/70; A61K 31/4748

(52) U.S. Cl. CPC A61K 9/7061; A61K 31/4748

(57) ABSTRACT

The present invention relates to a transdermal therapeutic system for the transdermal administration of huperzine A comprising a huperzine A-containing layer structure, said huperzine A-containing layer structure comprising A) a backing layer; and B) a huperzine A-containing layer.

### TECHNICAL FIELD OF THE INVENTION

The present invention relates to a transdermal therapeutic system (TTS) for the transdermal administration of huperzine A to the systemic circulation and uses thereof.

### BACKGROUND OF THE INVENTION

The active agent huperzine A (also known as (−)-Huperzine A, (−)-Selagine, (1R,9R, 13E)-1-amino-13-ethylidene-11-methyl-6-azatricyclo[7.3.1.02,7]trideca-2 (7),3,10-trien-5-one, C₁₅H₁₈N₂O, CAS No. 102518-79-6) is a naturally occurring sesquiterpene alkaloid found in the extracts of the firmoss Huperzia serrata and in varying quantities in other food Huperzia species, including H. elmeri, H. carinat, and H. aqualupian. It is a selective and reversible acetylcholinesterase (AChE) inhibitor as well as NMDA receptor antagonist that crosses the blood-brain barrier. It has the following chemical formula.

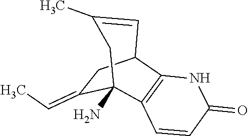 [SMILES: C/C=C1\CC2C=C(C)C[C@]1(N)c1ccc(=O)[nH]c12]

The botanical has been used in traditional Chinese medicine for centuries for the treatment of swelling, fever and blood disorders, and to enhance memory and concentration. For some years, huperzine A has been investigated as a possible treatment for diseases characterized by neurodegeneration, particularly Alzheimer's disease. Currently, huperzine A is commercially available in the form of tablets up to a daily oral dosage of pure huperzine A of 0.2 to 0.5 mg. Multiple tablets are taken throughout the day (up to 9 tablets).

However, the oral administration of active agents has disadvantages, e.g., in terms of patient compliance.

Therefore, a need exists for a transdermal therapeutic system for the transdermal administration of huperzine A. In particular, a need exists for a TTS, which is suitable for multi day therapy with a single application thereby improving patient compliance.

### OBJECTS AND SUMMARY OF THE INVENTION

It is therefore an object of the present invention to provide a TTS for the transdermal administration of huperzine A. Thus, it is an object of the present invention to provide a TTS for the transdermal administration of huperzine A providing a skin permeation rate which is sufficient for achieving a therapeutically effective dose. In particular, it is an object of the present invention to provide a matrix-type TTS for the transdermal administration of huperzine A providing a permeation rate which is sufficient for achieving a therapeutically effective dose.

It is a further object of the present invention to provide a TTS for the transdermal administration of huperzine A, wherein the fluctuation in huperzine A blood plasma concentration is reduced when compared to oral administration, in particular at steady state.

It is a further object of the present invention to provide a TTS for the transdermal administration of huperzine A with a high huperzine A utilization.

It is another object of the present invention to provide a TTS for the transdermal administration of huperzine A which complies with the needs of a convenient application in view of size and thickness and/or which is easy and cost-efficient to manufacture.

It is a further object of the present invention to provide a TTS for the transdermal administration of huperzine A providing therapeutically effective amounts of huperzine A for at least 24 hours (1 day), or at least 84 hours (3.5 days), or at least 168 h (7 days). In particular, it is an object of the present invention that the therapeutically effective amounts are provided over the whole time period, wherein the TTS is applied to the skin, allowing an around the clock treatment by exchanging the TTS after a certain application time, such as, e.g., after 24 h (once a day), after 84 hours (twice a week), or after 168 h (once a week).

These objects and others are accomplished by the present invention, which according to one aspect relates to a transdermal therapeutic system for the transdermal administration of huperzine A comprising a huperzine A-containing layer structure, said huperzine A-containing layer structure comprising:
A) a backing layer; and
B) a huperzine A-containing layer;
wherein said huperzine A-containing layer is a huperzine A-containing matrix layer comprising:
i) huperzine A; and
ii) at least one polymer selected from the group consisting of silicone polymers, polyisobutylenes, acrylate polymers and styrene-isoprene-styrene block copolymers; and
iii) at least one additive selected from crystallization inhibitors, permeation enhancers and combinations thereof.

It has been found that the TTS according to the present invention provides advantageous properties in terms of the utilization of huperzine A and the constant and continuous huperzine A delivery. In particular, the TTS according to the present invention provides suitable permeation rates and suitable permeated amounts of huperzine A over a time period of at least 24 hours, or at least 84 hours, or at least 168 hours. In certain preferred embodiments, the huperzine A-containing matrix layer is a self-adhesive layer structure and wherein the transdermal therapeutic system does not comprise an additional skin contact layer.

In certain preferred embodiments, the at least one polymer is an acrylate polymer or a styrene-isoprene-styrene block copolymer, wherein the acrylate polymer is preferably an acrylic copolymer comprising a hydroxyl group based on vinyl acetate, 2-ethylhexyl-acrylate and 2-hydroxyethyl-acrylate or wherein the polymer is preferably a styrene-isoprene-styrene block copolymer.

In certain preferred embodiments, the crystallization inhibitor is selected from polyvinylpyrrolidone (PVP, povidone), polyvinylpolypyrrolidone (PVPP, crospovidone) and combinations thereof.

In certain preferred embodiments, the permeation enhancer is selected from the group consisting of transcutol, lauryl lactate, methyl laurate, capryol 90, 1-dodecanol, levulinic acid and combinations thereof, and is preferably selected from methyl laurate, capryol 90, 1-dodecanol and combinations thereof.

It has surprisingly been found by the inventors of the present invention that a TTS comprising methyl laurate, 1-dodecanol, or a combination thereof as permeation enhancer shows an enhanced skin permeation rate and thus cumulative permeated amount of huperzine A. In particular, it has surprisingly been found that a TTS comprising methyl laurate, 1-dodecanol or combinations thereof in the huperzine A-containing layer shows an enhanced cumulative permeated amount of huperzine A over at least 72 h in comparison to a TTS comprising no permeation enhancer or other permeation enhancers in the huperzine A-containing layer. Further, it has surprisingly been found that the skin permeation rate and thus the cumulative permeated amount of huperzine A can be particularly increased upon using methyl laurate as permeation enhancer and even more increased upon using a combination of methyl laurate and 1-dodecanol as permeation enhancer.

Thus, in a preferred embodiment, the TTS according to the present invention, in particular the huperzine A-containing matrix layer comprises a permeation enhancer, wherein the permeation enhancer is selected from methyl laurate, 1-dodecanol, or combinations thereof.

In a particularly preferred embodiment, the TTS according to the present invention, in particular the huperzine A-containing matrix layer comprises a permeation enhancer, wherein the permeation enhancer is methyl laurate. In another particularly preferred embodiment, the TTS according to the present invention, in particular the huperzine A-containing matrix layer comprises a permeation enhancer, wherein the permeation enhancer is a combination of methyl laurate and 1-dodecanol.

In a more preferred embodiment, the transdermal therapeutic system comprises at least two additives, wherein the first additive is a crystallization inhibitor, preferably polyvinylpyrrolidone (PVP, povidone) or polyvinylpolypyrrolidone (PVPP, crospovidone), and the second additive is a permeation enhancer, preferably methyl laurate and/or 1-dodecanol.

According to a specific embodiment, the huperzine A-containing layer comprises:
i) huperzine A; and
ii) an acrylate polymer, preferably an acrylic copolymer comprising a hydroxyl group based on vinyl acetate, 2-ethylhexyl-acrylate, and 2-hydroxyethyl-acrylate; and
iii) at least two additives selected from methyl laurate and polyvinylpyrrolidone (PVP, povidone).

According to another specific embodiment, the huperzine A-containing layer comprises:
i) huperzine A
ii) at least one polymer selected from styrene-isoprene-styrene block copolymers; and
iii) at least two additives selected from methyl laurate and polyvinylpolypyrrolidone (PVPP, crospovidone).

According to another aspect, the invention further relates to the transdermal therapeutic system according to the invention for use in a method of treating Alzheimer's disease or any disease related to cognitive dysfunction. In connection with these medical uses, the TTS according to the invention is preferably applied to the skin of the patient for at least 24 hours, or 84 hours, or 168 hours.

### Definitions

Within the meaning of this invention, the term “transdermal therapeutic system” (TTS) refers to a system by which the active agent (e.g. huperzine A) is administered to the systemic circulation via transdermal delivery and refers to the entire individual dosing unit that is applied, after removing an optionally present release liner, to the skin of a patient, and which comprises a therapeutically effective amount of active agent in an active agent-containing layer structure and optionally an additional adhesive overlay on top of the active agent-containing layer structure. The active agent-containing layer structure may be located on a release liner (a detachable protective layer), thus, the TTS may further comprise a release liner. Within the meaning of this invention, the term “TTS” in particular refers to systems providing transdermal delivery, excluding active delivery for example via iontophoresis or microporation. Transdermal therapeutic systems may also be referred to as transdermal drug delivery systems (TDDS) or transdermal delivery systems (TDS).

Within the meaning of this invention, the term “active agent-containing layer structure” or “huperzine A-containing layer structure” refers to the layer structure comprising a backing layer and an active agent-containing layer. The active agent-containing layer structure comprises a therapeutically effective amount of an active agent. Preferably, the active agent-containing layer structure is an active agent-containing self-adhesive layer structure. Preferably, the active agent is huperzine A.

Within the meaning of this invention, the term “therapeutically effective amount” refers to a quantity of active agent in the TTS sufficient to provide, if administered by the TTS to a patient, the desired pharmacological effect. With regard to huperzine A, the term “therapeutically effective amount” preferably refers to a quantity of active agent in the TTS which is, if administered by the TTS to a patient, sufficient to treat, prevent or reduce Alzheimer's disease or any disease related to cognitive dysfunction. A TTS usually contains more active in the system than is in fact provided to the skin and the systemic circulation. This excess amount of active agent is usually necessary to provide enough driving force for the delivery from the TTS to the systemic circulation.

Within the meaning of this invention, the terms “active”, “active agent”, and the like, as well as the term “huperzine A” refer to huperzine A in any pharmaceutically acceptable chemical and morphological form and physical state. These forms include without limitation the active agent huperzine A in its free base form, protonated or partially protonated form, deprotonated or partially deprotonated form, salts, cocrystals, acid addition salts formed by addition of an inorganic or organic acid, solvates, hydrates, clathrates, complexes and so on, as well as the active agent in the form of particles, which may be micronized, crystalline and/or amorphous, and any mixtures of the aforementioned forms. The active agent, where contained in a medium such as a solvent, may be dissolved or dispersed or in part dissolved and in part dispersed. Preferably, huperzine A refers to (−)-huperzine A.

When the active agent is mentioned to be used in a particular form in the manufacture of the TTS, this does not exclude interactions between this form of the active agent and other ingredients of the active agent-containing layer structure. This means that the active agent may be present in the final TTS in protonated or partially protonated/or deprotonated or partially deprotonated form or in the form of an acid addition salt, or, if it is included in the form of a salt, parts of it may be present as free base in the final TTS. Unless otherwise indicated, in particular the amount of the active agent in the layer structure relates to the amount of the active agent included in the TTS during manufacture of the TTS and is calculated based on the active agent itself, but not on other forms thereof.

The active agent starting material included in the TTS during manufacture of the TTS may be in the form of particles. The active agent may e.g. be present in the active agent-containing layer structure in the form of particles and/or dissolved.

Within the meaning of this invention, the term “particles” refers to a solid, particulate material comprising individual particles, the dimensions of which are negligible compared to the material. In particular, the particles are solid, including plastic/deformable solids, including amorphous and crystalline materials.

Within the meaning of this invention, the term “dispersing” refers to a step or a combination of steps wherein a starting material (e.g. huperzine A) is not totally dissolved. Dispersing in the sense of the invention comprises the dissolution of a part of the starting material (e.g. huperzine A particles), depending on the solubility of the starting material (e.g. the solubility of huperzine A in the coating composition).

There are two main types of TTS for active agent delivery, i.e. matrix-type TTS and reservoir-type TTS. The release of the active agent in a matrix-type TTS is mainly controlled by the matrix including the active agent itself. In contrast thereto, a reservoir-type TTS typically needs a rate-controlling membrane controlling the release of the active agent. In principle, also a matrix-type TTS may contain a rate-controlling membrane. However, matrix-type TTS are advantageous in that, compared to reservoir-type TTS, usually no rate determining membranes are necessary and no dose dumping can occur due to membrane rupture. In summary, matrix-type transdermal therapeutic systems (TTS) are less complex in manufacture and easy and convenient to use by patients. Therefore, the TTS of the present invention is a matrix-type TTS, i.e. the active agent-containing layer (huperzine A-containing layer) is an active agent-containing matrix layer (huperzine A-containing matrix layer).

Within the meaning of this invention, “matrix-type TTS” refers to a system or structure wherein the active is homogeneously dissolved and/or dispersed within a polymeric carrier, i.e. the matrix, which forms with the active agent and optionally remaining ingredients a matrix layer. In such a system, the matrix layer controls the release of the active agent from the TTS. Preferably, the matrix layer has sufficient cohesion to be self-supporting so that no sealing between other layers is required. Accordingly, the active agent-containing layer may in one embodiment of the invention be an active agent-containing matrix layer, wherein the active agent is homogeneously distributed within a polymer matrix. In certain embodiments, the active agent-containing matrix layer may comprise two active agent-containing matrix layers, which may be laminated together. Matrix-type TTS may in particular be in the form of a “drug-in-adhesive”-type TTS referring to a system wherein the active is homogeneously dissolved and/or dispersed within a pressure-sensitive adhesive matrix. In this connection, the active agent-containing matrix layer may also be referred to as active agent-containing pressure sensitive adhesive layer or active agent-containing pressure sensitive adhesive matrix layer. A TTS comprising the active agent dissolved and/or dispersed within a polymeric gel, e.g. a hydrogel, is also considered to be of matrix-type in accordance with present invention. It is to be understood that a TTS comprising an active agent-containing matrix layer may additionally also comprise a skin contact layer.

TTS with a liquid active agent-containing reservoir are referred to by the term “reservoir-type TTS”. In such a system, the release of the active agent is preferably controlled by a rate-controlling membrane. In particular, the reservoir is sealed between the backing layer and the rate-controlling membrane. Accordingly, the active agent-containing layer may in one embodiment be an active agent-containing reservoir layer, which preferably comprises a liquid reservoir comprising the active agent. Furthermore, the reservoir-type TTS typically additionally comprises a skin contact layer, wherein the reservoir layer and the skin contact layer may be separated by the rate-controlling membrane. In the reservoir layer, the active agent is preferably dissolved in a solvent such as ethanol or water or in silicone oil. The skin contact layer typically has adhesive properties.

Reservoir-type TTS are not to be understood as being of matrix-type within the meaning of the invention.

Within the meaning of this invention, the term “active agent-containing layer” refers to a layer containing the active agent and providing the area of release. If the active agent-containing layer is an active agent-containing matrix layer, said layer is present in a matrix-type TTS. In the present invention, the huperzine A-containing layer is an huperzine A-containing matrix layer, i.e., said layer is present in a matrix-type TTS. If the polymer is a pressure-sensitive adhesive, the matrix layer may also represent the adhesive layer of the TTS, so that no additional skin contact layer is present. Alternatively, an additional skin contact layer may be present as adhesive layer, and/or an adhesive overlay is provided. The additional skin contact layer is typically manufactured such that it is active agent-free. However, due to the concentration gradient, the active agent will migrate from the matrix layer to the additional skin contact layer over time, until an equilibrium is reached. The additional skin contact layer may be present on the active agent-containing matrix layer or separated from the active agent-containing matrix layer by a membrane, preferably a rate controlling membrane. Preferably, the active agent-containing matrix layer has sufficient adhesive properties, so that no additional skin contact layer is present. If the active agent-containing layer is an active agent-containing reservoir layer, said layer is present in a reservoir-type TTS, and the layer comprises the active agent in a liquid reservoir. In addition, a skin contact layer may be present, in order to provide adhesive properties. The additional skin contact layer is typically manufactured such that it is active agent-free. If the skin contact layer is free of active agent the active agent will migrate, due to the concentration gradient, from the reservoir layer to the skin contact layer over time, until an equilibrium is reached. Additionally, an adhesive overlay may be provided.

As used herein, the active agent-containing layer is an active agent-containing matrix layer, and it is referred to the final solidified layer. Preferably, an active agent-containing matrix layer is obtained after coating and drying the solvent-containing coating composition as described herein. Alternatively, an active agent-containing matrix layer is obtained after melt-coating and cooling. The active agent-containing matrix layer may also be manufactured by laminating two or more such solidified layers (e.g. dried or cooled layers) of the same composition to provide the desired area weight. Preferably, the matrix layer is a pressure sensitive adhesive matrix layer. Optionally, an adhesive overlay may be present.

Within the meaning of this invention, the term “pressure-sensitive adhesive” (also abbreviated as “PSA”) refers to a material that in particular adheres with finger pressure, is permanently tacky, exerts a strong holding force and should be removable from smooth surfaces without leaving a residue. A pressure sensitive adhesive layer, when in contact with the skin, is “self-adhesive”, i.e. provides adhesion to the skin so that typically no further aid for fixation on the skin is needed. A “self-adhesive” layer structure according to the present invention includes a pressure sensitive adhesive layer for skin contact which may be provided in the form of a pressure sensitive matrix layer or in the form of an additional layer, i.e. a pressure sensitive adhesive skin contact layer. Preferably, no additional skin contact layer is present and the self-adhesive layer structure according to the present invention is a pressure sensitive adhesive matrix layer. An adhesive overlay may still be employed to advance adhesion. The pressure-sensitive adhesive properties of a pressure-sensitive adhesive depend on the polymer or polymer composition used.

As used herein, an “acrylate polymer” or “acrylic polymer” refers to a polymer obtainable from one or more monomers selected from acrylic acid, butylacrylate, 2-ethylhexylacrylate, glycidylmethacrylate, 2-hydroxyethylacrylate, methylacrylate, methylmethacrylate, butylmethacrylate, t-octylacrylamide, and vinylacetate.

As used herein, a “silicone polymer” refers to polymers based on polysiloxanes. They may therefore also be referred to as silicone-based polymers or polymers based on polysiloxanes. Preferably, the silicone-based polymers are silicone-based pressure sensitive adhesives. Silicone-based pressure sensitive adhesives may also be referred to as silicone pressure sensitive adhesives or pressure-sensitive adhesives based on polysiloxanes.

As used herein, a “styrene-isoprene-styrene block copolymer” or “SIS” or “SIS block copolymer” refers to a polymer obtainable from copolymerization of the monomers styrene and isoprene, i.e. styrene and 2-methyl-1,3-butadiene. In particular, it refers to a triblock copolymer with three distinct blocks, namely two blocks of polymerized styrene, separated by one block of polymerized isoprene.

As used herein, a “polyisobutylene” polymer refers to a pressure-sensitive adhesive based on polyisobutylenes. Pressure-sensitive adhesives based on polyisobutylenes may also be referred to as polyisobutylene-based pressure-sensitive adhesives. Suitable polyisobutylenes according to the invention, in particular pressure-sensitive adhesives based on polyisobutylenes are combinations of high-molecular weight polyisobutylenes and low-molecular weight polyisobutylenes. Suitable ratios of low-molecular weight polyisobutylene to high-molecular weight polyisobutylene are in the range of from 100:1 to 1:100, preferably from 95:5 to 40:60, more preferably from 90:10 to 80:20.

As used herein, a “permeation enhancer” refers to an additive, which increases the skin flux and permeation of the active ingredient. Furthermore, as used herein a “crystallization inhibitor” relates to an additive, which increases the solubility of the active ingredient in the active ingredient-containing layer.

As used herein, an active agent-containing matrix layer is a layer containing the active agent dissolved or dispersed in at least one polymer, or containing the active agent dissolved in a solvent to form an active agent-solvent mixture that is dispersed in the form of deposits (in particular droplets) in at least one polymer. Preferably, the at least one polymer is a polymer-based pressure-sensitive adhesive (e.g. an acrylic polymer). Within the meaning of this invention, the term “pressure-sensitive adhesive layer” refers to a pressure-sensitive adhesive layer obtained from a solvent-containing adhesive coating composition after coating on a film and evaporating the solvents.

Within the meaning of this invention, the term “skin contact layer” refers to the layer included in the active agent-containing layer structure to be in direct contact with the skin of the patient during administration. When the TTS comprises a skin contact layer, the other layers of the active agent-containing layer structure do not contact the skin and do not necessarily have self-adhesive properties. As outlined above, an additional skin contact layer attached to the active agent-containing layer may over time absorb parts of the active agent. The sizes of the skin contact layer and the active agent-containing layer are usually coextensive and correspond to the area of release. However, the area of the skin contact layer may also be greater than the area of the active agent-containing layer. In such a case, the area of release still refers to the area of the active agent-containing layer.

Within the meaning of this invention, the term “area weight” refers to the dry weight of a specific layer, e.g. of the matrix layer, provided in g/m². The area weight values are subject to a tolerance of +10%, preferably +7.5%, due to manufacturing variability.

As used herein, the term API-loading refers to the loading of the transdermal therapeutic system according to the present invention with the active pharmaceutical ingredient (API), i.e. according to the present invention to the huperzine A loading of the TTS.

If not indicated otherwise “%” refers to weight-% (% by weight).

Within the meaning of this invention, the term “polymer” refers to any substance consisting of so-called repeating units obtained by polymerizing one or more monomers, and includes homopolymers which consist of one type of monomer and copolymers which consist of two or more types of monomers. Polymers may be of any architecture such as linear polymers, star polymer, comb polymers, brush polymers, of any monomer arrangements in case of copolymers, e.g. alternating, statistical, block copolymers, or graft polymers. The minimum molecular weight varies depending on the polymer type and is known to the skilled person. Polymers may e.g. have a molecular weight above 2000, preferably above 5000 and more preferably above 10,000 Dalton. Correspondingly, compounds with a molecular weight below 2000, preferably below 5000 or more preferably below 10,000 Dalton are usually referred to as oligomers.

Within the meaning of this invention, the term “cross-linking agent” refers to a substance which is able to cross-link functional groups contained within the polymer.

Within the meaning of this invention, the term “adhesive overlay” refers to a self-adhesive layer structure that is free of active agent and larger in area than the active agent-containing structure and provides additional area adhering to the skin, but no area of release of the active agent. It enhances thereby the overall adhesive properties of the TTS. The adhesive overlay comprises a backing layer that may provide occlusive or non-occlusive properties and an adhesive layer. Preferably, the backing layer of the adhesive overlay provides non-occlusive properties.

Within the meaning of this invention, the term “backing layer” refers to a layer which supports the active agent-containing layer or forms the backing of the adhesive overlay. At least one backing layer in the TTS and usually the backing layer of the active agent-containing layer is substantially impermeable to the active agent contained in the layer during the period of storage and administration and thus prevents active loss or cross-contamination in accordance with regulatory requirements. Preferably, the backing layer is also occlusive, meaning substantially impermeable to water and water-vapor. Suitable materials for a backing layer include polyethylene terephthalate (PET), polyethylene (PE), ethylene vinyl acetate-copolymer (EVA), polyurethanes, and mixtures thereof. Suitable backing layers are thus for example PET laminates, EVA-PET laminates and PE-PET laminates. Also suitable are woven or non-woven backing materials.

The TTS according to the present invention can be characterized by certain parameters as measured in an in vitro skin permeation test.

In general, the in vitro permeation test is performed in a Franz diffusion cell, with 0.9% sodium chloride solution with 0.1% sodium azide as antibacteriological agent.

Further, in vitro permeation test may be performed in a Franz diffusion cell, with human or animal skin and preferably with dermatomed Göttinger minipig with a thickness of around 800 μm and an intact epidermis, and with 0.9% sodium chloride solution (32° C. with 0.1% saline azide).

Where not otherwise indicated, the in vitro permeation test is performed with dermatomed Göttinger minipig skin with a thickness around 800 μm and an intact epidermis, and with 0.9% Sodium chloride solution as receptor medium (32° C. with 0.1% saline azide). The amount of active permeated into the receptor medium is determined in regular intervals using a HPLC method) with a UV photometric detector by taking a sample volume. The receptor medium is completely or in part replaced by fresh medium when taking the sample volume, and the measured amount of active permeated relates to the amount permeated between the two last sampling points and not the total amount permeated so far.

Thus, within the meaning of this invention, the parameter “permeated amount” is provided in μg/cm² and relates to the amount of active agent permeated in a sample interval at certain elapsed time. E.g., in an in vitro permeation test as described above, wherein the amount of active agent permeated into the receptor medium has been e.g. measured at hours 0, 4, 8, 16, and 24, the “permeated amount” of active can be given e.g. for the sample interval from hour 8 to hour 16 and corresponds to the measurement at hour 16, wherein the receptor medium has been exchanged completely at hour 8.

The permeated amount can also be given as a “cumulative permeated amount”, corresponding to the cumulated amount of active agent permeated at a certain point in time. E.g., in an in vitro permeation test as described above, wherein the amount of active agent permeated into the receptor medium has been e.g. measured at hours 0, 4, 8, 16 and 24, the “cumulative permeated amount” of active agent at hour 16 corresponds to the sum of the permeated amounts from hour 0 to hour 4, hour 4 to hour 8 and hour 8 to hour 16.

Within the meaning of this invention, the parameter “skin permeation rate” for a certain sample interval at certain elapsed time is provided in μg/(cm²*h) and is calculated from the permeated amount in said sample interval as measured by in vitro permeation test as described above in μg/cm², divided by the hours of said sample interval. E.g. the skin permeation rate in an in vitro permeation test as described above, wherein the amount of active permeated into the receptor medium has been e.g. measured at hours 0, 4, 8, 16 and 24, the “skin permeation rate” at hour 16 is calculated as the permeated amount in the sample interval from hour 8 to hour 16 divided by 8 hours.

A “cumulative skin permeation rate” can be calculated from the respective cumulative permeated amount by dividing the cumulative permeated amount by the elapsed time. E.g. in an in vitro permeation test as described above, wherein the amount of active permeated into the receptor medium has been e.g. measured at hours 0, 4, 8, 16 and 24, the “cumulative skin permeation rate” at hour 16 is calculated as the cumulative permeated amount for hour 16 (see above) divided by 16 hours.

Within the meaning of this invention, the above parameters “permeated amount” and “skin permeation rate” (as well as “cumulative permeated amount” and “cumulative skin permeation rate”) refer to mean values calculated from at least 2 in vitro permeation test experiments. Where not otherwise indicated, the standard deviation (SD) of these mean values refer to a corrected sample standard deviation, calculated using the formula:

$$SD = \sqrt{\frac{1}{n - 1} {\sum }_{i = 1}^{n} {( {x}_{i} - \overline{x} )}^{2}}$$

wherein n is the sample size, {x₁, x₂, . . . , xₙ} are the observed values and x̅ is the mean value of the observed values.

The TTS according to the present invention can also be characterized by certain parameters as measured in an in vivo clinical study.

Within the meaning of this invention, the parameter “mean release rate” refers to the mean release rate in μg/h (μg/hour) or in mg/day over the period of administration (e.g., 1 to 7 days) by which the active agent is released through the human skin into the systemic circulation and is based on the AUC obtained over said period of administration in a clinical study.

Within the meaning of this invention, the term “extended period of time” relates to a period of at least or about 24 hours, at least or about 48 hours, at least or about 84 hours, at least or about 168 hours, at least or about 1 day, at least or about 2 days, at least or about 3.5 days, or at least or about 7 days, or to a period of about 24 hours to about 168 hours or 1 to 7 day(s), or about 24 hours to about 84 hours or 1 to 3.5 day(s).

For a continuous drug treatment, the frequency of drug administration is preferably kept sufficiently high so as to maintain therapeutically effective blood plasma concentration. In other words, the interval between two dosage form administrations, also called dosing interval, needs to be adapted accordingly. Within the meaning of the present invention, the term “dosing interval” refers to the period of time between two consecutive TTS administrations, i.e. the interval between two consecutive points in time a TTS is applied to the skin of the patient. Once applied, the TTS is usually maintained on the skin of the patient for the entire dosing interval and only removed at the end of the dosing interval, at which time a new TTS is applied to the skin. E.g., if the dosing interval is 24 hours or 1 day, the TTS is applied to and maintained on the skin of the patient for 24 hours or 1 day. After 24 hours or 1 day, the TTS is removed from the skin and a new TTS is applied. Thus, a dosing interval of 24 hours or 1 day allows a daily TTS exchange mode in an around-the-clock treatment.

Within the meaning of this invention, the term “room temperature” refers to the unmodified temperature found indoors in the laboratory where the experiments are conducted and usually lies within 15 to 35° C., preferably about 18 to 25° C.

Within the meaning of this invention, the term “patient” refers to a subject who has presented a clinical manifestation of a particular symptom or symptoms suggesting the need for treatment, who is treated preventatively or prophylactically for a condition, or who has been diagnosed with a condition to be treated.

Within the meaning of this invention the term “pharmacokinetic parameters” refers to parameters describing the blood plasma curve, e.g. Cₘₐₓ, Cₜ and AUCₜ₁₋ₜ₂ obtained in a clinical study, e.g. by single-dose, multi-dose or steady state administration of the active agent-containing TTS, e.g. the guanfacine-containing TTS to healthy human subjects. The pharmacokinetic parameters of the individual subjects are summarized using arithmetic and geometric means, e.g. a mean Cₘₐₓ, a mean AUCt and a mean AUC_{INF}, and additional statistics such as the respective standard deviations and standard errors, the minimum value, the maximum value, and the middle value when the list of values is ranked (Median). In the context of the present invention, pharmacokinetic parameters, e.g. the Cₘₐₓ, Cₜ and AUCₜ₁₋ₜ₂ refer to geometric mean values if not indicated otherwise. It cannot be precluded that the absolute mean values obtained for a certain TTS in a clinical study vary to a certain extent from study to study. To allow a comparison of absolute mean values between studies, a reference formulation, e.g. in the future any product based on the invention, may be used as internal standard. A comparison of the AUC per area of release of the respective reference product in the earlier and later study can be used to obtain a correction factor to take into account differences from study to study.

Clinical studies according to the present invention refer to studies performed in full compliance with the International Conference for Harmonization of Clinical Trials (ICH) and all applicable local Good Clinical Practices (GCP) and regulations.

Within the meaning of this invention, the term “healthy human subject” refers to a male or female subject with a body weight ranging from 55 kg to 100 kg and a body mass index (BMI) ranging from 18 to 29.4 and normal physiological parameters, such as blood pressure, etc. Healthy human subjects for the purposes of the present invention are selected according to inclusion and exclusion criteria, which are based on and in accordance with recommendations of the ICH.

Within the meaning of this invention, the term “subject population” refers to at least five, preferably at least ten individual healthy human subjects.

Within the meaning of this invention, the term “geometric mean” refers to the mean of the log transformed data back-transformed to the original scale.

Within the meaning of this invention, the term “arithmetic mean” refers to the sum of all values of observation divided by the total number of observations.

Within the meaning of this invention, the parameter “AUC” corresponds to the area under the plasma concentration-time curve. The AUC value is proportional to the amount of active agent absorbed into the blood circulation in total and is hence a measure for the bioavailability.

Within the meaning of this invention, the parameter “AUCₜ₁₋ₜ₂” is provided in (ng/ml) h and relates to the area under the plasma concentration-time curve from hour t1 to t2 and is calculated by the linear trapezoidal method, unless otherwise indicated. Other calculation methods are e.g. the logarithmic and linear log trapezoidal method.

Within the meaning of this invention, the parameter “Cₘₐₓ” is provided in (ng/ml) and relates to the maximum observed blood plasma concentration of the active agent.

Within the meaning of this invention, the parameter “Cₜ” is provided in (ng/ml) and relates to the blood plasma concentration of the active agent observed at hour t.

Within the meaning of this invention, the parameter “tₘₐₓ” is provided in hours and relates to the time point at which the Cₘₐₓ value is reached. In other words, tₘₐₓ is the time point of the maximum observed plasma concentration.

Within the meaning of this invention, the term “mean plasma concentration” is provided in (ng/ml) and is a mean of the individual plasma concentrations of active agent, e.g. guanfacine, at each point in time.

Within the meaning of this invention, the term “coating composition” refers to a composition comprising all components of the matrix layer in a solvent, which may be coated onto the backing layer or release liner to form the matrix layer upon drying.

Within the meaning of this invention, the term “pressure sensitive adhesive composition” refers to a pressure sensitive adhesive at least in mixture with a solvent (e.g. n-heptane or ethyl acetate).

Within the meaning of this invention, the term “dissolve” refers to the process of obtaining a solution, which is clear and does not contain any particles, as visible to the naked eye.

Within the meaning of this invention, the term “solvent” refers to any liquid substance, which preferably is a volatile organic liquid such as methanol, ethanol, isopropanol, acetone, ethyl acetate, methylene chloride, hexane, n-heptane, toluene and mixtures thereof.

Within the meaning of this invention, the term “equimolar” refers to the amount in which two or more ingredients are present. Preferably, “equimolar” means that the ingredients are present in a ratio of 1:1.

### BRIEF DESCRIPTION OF THE DRAWINGS

FIG. 1A-C depict the huperzine A cumulative permeated amounts of TTS prepared according to Examples 1A-F and the utilization of huperzine A of TTS prepared according to Examples 1A-F after 72 h.

FIG. 2A-C depict the huperzine A cumulative permeated amounts of TTS prepared according to Examples 2A-E and the utilization of huperzine A of TTS prepared according to Examples 2A-E after 72 h.

FIG. 3A-C depict the huperzine A cumulative permeated amounts of TTS prepared according to Examples 3A-E and the utilization of huperzine A of TTS prepared according to Examples 3A-E after 72 h.

FIG. 4A-C depict the huperzine A cumulative permeated amounts of TTS prepared according to Examples 4A-F and the utilization of huperzine A of TTS prepared according to Examples 4A-F after 168 h.

FIG. 5A-C depict the huperzine A cumulative permeated amounts of TTS prepared according to Examples 5A-F and the utilization of huperzine A of TTS prepared according to Examples 5A-F after 168 h.

FIG. 6A-C depict the huperzine A cumulative permeated amounts of TTS prepared according to Examples 6A-E and comparative Examples 1A and 1B as well as the utilization of huperzine A of TTS prepared according to Examples 6A-E and comparative Examples 1A and 1B after 72 h.

### DETAILED DESCRIPTION

### TTS Structure

The present invention is related to a transdermal therapeutic system for the transdermal administration of huperzine A comprising a huperzine A-containing layer structure. The huperzine A-containing layer structure in particular may contain a therapeutically effective amount of huperzine A.

Thus, in a first aspect, the present invention relates to a transdermal therapeutic system for the transdermal administration of huperzine A comprising a huperzine A-containing layer structure, said huperzine A-containing layer structure comprising:
A) a backing layer; and
B) a huperzine A-containing layer;
wherein said huperzine A-containing layer is a huperzine A-containing matrix layer comprising:
i) huperzine A; and
ii) at least one polymer selected from the group consisting of silicone polymers, polyisobutylenes, acrylate polymers and styrene-isoprene-styrene block copolymers; and
iii) at least one additive selected from crystallization inhibitors, permeation enhancers and combinations thereof.

The huperzine A-containing layer structure is preferably a huperzine A-containing self-adhesive layer structure. Preferably, the at least one polymer in the huperzine A-containing layer provides the self-adhesive properties. In this connection, it is not required that the huperzine A-containing layer structure comprises an additional skin contact layer. Thus, the huperzine A-containing layer structure, preferably the huperzine A-containing self-adhesive layer structure, preferably does not comprise an additional skin contact layer.

The TTS according to the present invention is a matrix-type TTS. In such a matrix-type TTS according to the invention, the huperzine A is homogeneously dissolved and/or dispersed within a polymeric carrier, i.e. the matrix, which forms with the huperzine A and optionally additional ingredients a matrix layer. Thus, the huperzine A-containing layer according to the present invention is a huperzine A-containing matrix layer. While the huperzine A-containing layer structure may or may not comprise an additional skin contact layer, it is preferred that the huperzine A-containing matrix layer is self-adhesive, so that an additional skin contact layer is not required and preferably is not present. If a huperzine A-containing matrix layer is prepared by laminating together two huperzine A-containing matrix layers, which are of substantially the same composition, the resulting double layer is to be regarded as one huperzine A-containing matrix layer.

As outlined above, in a preferred embodiment, the huperzine A-containing layer structure is a huperzine A-containing self-adhesive layer structure and preferably does not comprise an additional skin contact layer. In particular, the huperzine A-containing layer structure is a huperzine A-containing matrix layer and is a self-adhesive layer structure and wherein the transdermal therapeutic system does not comprise an additional skin contact layer.

Alternatively or additionally, it is preferred that the huperzine A-containing layer is directly attached to the backing layer, so that there is no additional layer between the backing layer and the huperzine A-containing layer. Consequently, a layer structure of low complexity is obtained, which is advantageous, e.g., in terms of the costs for the manufacture.

In particular, it is preferred that the huperzine A-containing layer structure comprises not more than 3 and preferably comprises 2 layers, i.e. only the backing layer and the huperzine A-containing layer. Sufficient adhesion between the huperzine A-containing layer structure and the skin of the patient during administration is then provided by the huperzine A-containing matrix layer, which is preferably a huperzine A-containing self-adhesive matrix layer, more preferably a huperzine A-containing pressure-sensitive adhesive matrix layer.

If an additional skin contact layer is present, e.g., as the third layer of the huperzine A-containing layer structure, the adhesive properties may be provided by the additional skin contact layer such that the additional skin contact layer is preferably self-adhesive and provides for adhesion between the huperzine A-containing layer structure and the skin of the patient during administration. However, it is preferred according to the invention that no additional skin contact layer is present.

The self-adhesive properties of the huperzine A-containing layer structure are preferably provided by the at least one polymer selected from the group consisting of silicone polymers, polyisobutylenes, acrylate polymers and styrene-isoprene-styrene block copolymers, which is present in the huperzine A-containing matrix layer. Further details regarding the at least one polymer in the huperzine A-containing matrix layer are provided further below.

It is to be understood that the TTS according to the invention, in particular the huperzine A-containing matrix layer, further comprises at least one additive selected from the group consisting of crystallization inhibitors, permeation enhancers and combinations thereof. Further details regarding the at least one additive in the huperzine A-containing matrix layer are provided further below.

In a preferred embodiment of the invention, the backing layer is substantially impermeable for huperzine A. Furthermore, it is preferred that the backing layer is occlusive as outlined above.

According to certain embodiments of the invention, the TTS may further comprise an adhesive overlay. This adhesive overlay is in particular larger in area than the huperzine A-containing layer structure and is attached thereto for enhancing the adhesive properties of the overall transdermal therapeutic system. Said adhesive overlay comprises a backing layer and an adhesive layer. The adhesive overlay provides additional area adhering to the skin but does not add to the area of release of the huperzine A. The adhesive overlay comprises a self-adhesive polymer or a self-adhesive polymer mixture selected from the group consisting of silicone acrylic hybrid polymers, acrylate polymers, silicone polymers, polyisobutylenes, styrene-isoprene-styrene copolymers, and mixtures thereof, which may be identical to or different from any polymer or polymer mixture included in the huperzine A-containing layer structure.

The huperzine A-containing layer structure according to the invention, such as a huperzine A-containing self-adhesive layer structure, is normally located on a detachable protective layer (release liner), from which it is removed immediately before application to the surface of the patient's skin. Thus, the TTS may further comprise a release liner. A TTS protected this way is usually stored in a blister pack or a seam-sealed pouch. The packaging may be child resistant and/or senior friendly.

Huperzine a-Containing Layer

As outlined above, the TTS according to the present invention comprises a huperzine A-containing layer, which is a huperzine A-containing matrix layer comprising
i) huperzine A; and
ii) at least one polymer selected from the group consisting of silicone polymers, polyisobutylenes, acrylate polymers and styrene-isoprene-styrene block copolymers; and
iii) at least one additive selected from crystallization inhibitors, permeation enhancers and combinations thereof.

Preferably, the huperzine A-containing layer structure is a huperzine A-containing self-adhesive layer structure. Accordingly, it is also preferred that the huperzine A-containing matrix layer is a self-adhesive huperzine A-containing matrix layer. Preferably, the huperzine A-containing matrix layer is a self-adhesive layer structure and wherein the transdermal therapeutic system does not comprise an additional skin contact layer. In a preferred embodiment, the huperzine A-containing matrix layer comprises a therapeutically effective amount of the huperzine A.

In order to maintain a certain driving force and thus to achieve sufficient skin permeation, the huperzine A-amount and concentration in the huperzine A-containing matrix layer is preferably kept at a certain level. Thus, in certain embodiments, the huperzine A-containing layer structure comprises huperzine A in an amount of from 1 to 10% by weight, e.g. from 1 to 3% by weight or from 4 to 10% by weight, preferably from 1.5 to 6% by weight, based on the total weight of the huperzine A-containing layer. In one embodiment, the amount of huperzine A in the huperzine A-containing matrix layer ranges from 1 to 3% by weight, preferably from 1.5 to 2.5% by weight, based on the total weight of the huperzine A-containing layer. In another embodiment, the amount of huperzine A in the huperzine A-containing matrix layer ranges from 4 to 10% by weight, preferably from 4 to 8% by weight, more preferably from 4.5 to 6% by weight, based on the total weight of the huperzine A-containing layer. In other preferred embodiment, the huperzine A containing layer has an API-loading, i.e. a huperzine A loading, of 150 to 1200 μg/cm², preferably from 200 to 800 g/cm², more preferably from 180 to 400 μg/cm² or from 450 to 800 μg/cm² or from 900 to 1100 μg/cm². The skilled person is aware on how to adapt or select the amount or concentration of huperzine A in the huperzine A-containing matrix layer depending on the desired dosing strength and dosing interval of the TTS.

In certain embodiments, the area weight of the huperzine A-containing layer ranges from 60 to 200 g/m², preferably from 70 to 150 g/m², and/or the huperzine A containing layer has an API-loading of 150 to 1200 μg/cm², preferably from 180 to 400 μg/cm² or from 450 to 800 μg/cm² or from 900 to 1100 μg/cm².

As indicated above, the TTS according to the present invention comprises at least one polymer in the huperzine A-containing layer, in particular in the huperzine A-containing matrix layer, wherein the at least one polymer is selected from the group consisting of silicone polymers, polyisobutylenes, acrylate polymers and styrene-isoprene-styrene block copolymers.

In certain embodiments, the at least one polymer is present in an amount of from 65 to 90% by weight, preferably from 70 to 85% by weight, even more preferably from 75 to 80% by weight, based on the total weight of the huperzine A-containing layer. Further details on the at least one polymer are provided further below.

As indicated above, the TTS according to the invention, and in particular the huperzine A-containing matrix layer, comprises at least one additive selected from crystallization inhibitors, permeation enhancers and combinations thereof.

In one embodiment, the at least one additive is a permeation enhancer. By adding a permeation enhancer, the skin flux and permeation can be increased. Thus, in a preferred embodiment, the huperzine A-containing layer comprises at least one, preferably one or two, permeation enhancer.

It has surprisingly been found by the inventors of the present invention that a TTS comprising methyl laurate, 1-dodecanol, or a combination thereof as permeation enhancer shows an enhanced skin flux rate and cumulative permeated amount of huperzine A compared to a TTS without these permeation enhancers or with other permeation enhancers. Thus, in a preferred embodiment, the at least one additive is a permeation enhancer selected from methyl laurate, 1-dodecanol, or combinations thereof, preferably methyl laurate or a combination of methyl laurate and 1-dodecanol.

In connection with the permeation enhancer in the huperzine A-containing matrix layer according to the invention, the permeation enhancer is preferably present in an amount of from 2 to 15% by weight, preferably from 2.5 to 7.5% by weight or from 7.5 to 12.5% by weight, based on the total weight of the huperzine A-containing layer.

In another embodiment, the at least one additive is a crystallization inhibitor. By adding a crystallization enhancer, the solubility of huperzine A in the huperzine A-containing matrix layer, in particular in the at least one polymer in the huperzine A-containing matrix layer can be increased. Thus, in a preferred embodiment, the huperzine A-containing layer comprises at least one, preferably one or two, more preferably one, crystallization inhibitor.

In connection with the crystallization inhibitor in the huperzine A-containing matrix layer according to the invention, the crystallization inhibitor is preferably present in an amount of from 5 to 25% by weight, preferably from 5 to 15% by weight or from 15 to 25% by weight, more preferably from 8 to 12% by weight or from 18 to 22% by weight, based on the total weight of the huperzine A-containing layer.

In certain embodiments, combinations of the afore-mentioned additives are preferred. Thus, in preferred embodiments, the huperzine A-containing matrix layer comprises at least two additives, wherein the first additive is a crystallization inhibitor and the second additive is a permeation inhibitor. In particular, in certain preferred embodiments, the huperzine A-containing layer comprises at least one, preferably one or two, permeation enhancers and at least one, preferably one, crystallization inhibitor.

In certain preferred embodiments, the transdermal therapeutic system comprises at least two additives, wherein the first additive is a crystallization inhibitor, preferably polyvinylpyrrolidone (PVP, povidone) or polyvinylpolypyrrolidone (PVPP, crospovidone), and the second additive is a permeation enhancer, preferably methyl laurate and/or 1-dodecanol.

Further details on the at least one additive, in particular the at least one permeation enhancer and/or the at least one crystallization inhibitor are provided further below.

In view of the above, in one embodiment of the present invention, the huperzine A-containing layer is a huperzine A-containing matrix layer comprising:
i) huperzine A; and
ii) an acrylate polymer, preferably an acrylic copolymer comprising a hydroxyl group based on vinyl acetate, 2-ethylhexyl-acrylate, and 2-hydroxyethyl-acrylate; and
iii) at least two additives, preferably selected from methyl laurate and polyvinylpyrrolidone (PVP, povidone).

In another embodiment of the present invention, the huperzine A-containing layer is a huperzine A-containing matrix layer comprising:
i) huperzine A; and
ii) an acrylate polymer, preferably an acrylic copolymer comprising a hydroxyl group based on vinyl acetate, 2-ethylhexyl-acrylate, and 2-hydroxyethyl-acrylate; and
iii) at least two additives, preferably selected from a combination of methyl laurate and 1-dodecanol and polyvinylpyrrolidone (PVP, povidone).

In another embodiment, the huperzine A-containing layer is a huperzine A-containing matrix layer comprising:
i) huperzine A
ii) at least one polymer selected from styrene-isoprene-styrene block copolymers; and
iii) at least two additives selected from methyl laurate and polyvinylpolypyrrolidone (PVPP, crospovidone).

In another embodiment, the huperzine A-containing layer is a huperzine A-containing matrix layer comprising:
i) huperzine A
ii) at least one polymer selected from styrene-isoprene-styrene block copolymers, wherein the styrene-isoprene-styrene block copolymer preferably contains 10 to 25% by weight of styrene, more preferably 15% by weight of styrene; and
iii) at least two additives, preferably selected from methyl laurate and polyvinylpolypyrrolidone (PVPP, crospovidone).

Further details regarding the huperzine A, the polymers and the additives in the TTS, in particular in the huperzine A-containing matrix layer of the TTS, according to the invention and the particular embodiments described above, are provided below.

### Huperzine A

The transdermal therapeutic system of the present invention comprises huperzine A, and in particular therapeutically effective amounts of huperzine A, in a huperzine A-containing layer structure, i.e. in a huperzine A-containing layer, in particular a huperzine A-containing matrix layer. While the active agent may, in accordance with the present invention, be present in the TTS in protonated or in free base form, the free base form is preferred.

Thus, in certain embodiments, the huperzine A in the huperzine A-containing layer is included in the form of the free base.

In certain embodiments, the huperzine A-containing matrix layer is obtainable by incorporating the huperzine A in the form of the free base.

In particular, at least 90 mol %, preferably at least 95 mol %, more preferably at least 98 mol % and most preferably at least 99 mol % of the huperzine A in the huperzine A-containing layer is present in the form of the free base.

The huperzine A in the huperzine A-containing matrix layer may be completely dissolved, or the huperzine A-containing layer may contain huperzine A particles, preferably constituted of huperzine A free base.

The amount of huperzine A in the TTS is important for the amount of active released and also for the release rate.

In certain embodiments, the amount of huperzine A in the huperzine A-containing matrix layer ranges from 1 to 10% by weight, e.g. from 1 to 3% by weight or from 4 to 10% by weight, preferably from 1.5 to 6% by weight, based on the total weight of the huperzine A-containing layer. In one embodiment, the amount of huperzine A in the huperzine A-containing matrix layer ranges from 1 to 3% by weight, preferably from 1.5 to 2.5% by weight, based on the total weight of the huperzine A-containing layer. In another embodiment, the amount of huperzine A in the huperzine A-containing matrix layer ranges from 4 to 10% by weight, preferably from 4 to 8% by weight, more preferably from 4.5 to 6% by weight, based on the total weight of the huperzine A-containing layer.

In certain embodiments, the area weight of the huperzine A-containing layer ranges from 60 to 200 g/m², preferably from 70 to 150 g/m², and/or the huperzine A containing layer has an API-loading of 150 to 1200 μg/cm², preferably from 180 to 400 μg/cm² or from 450 to 800 μg/cm² or from 900 to 1100 μg/cm².

The skilled person knows how to adapt the amount of huperzine A in the TTS, in particular in the huperzine A-containing matrix layer, depending on the release rate of the TTS and the desired dosing strength and dosing interval. For example, the amount of huperzine A in the TTS may be lower in a TTS for use in a method of treatment with a dosing interval of 24 hours or 1 day (an around-the-clock treatment with a once-a-day TTS exchange mode) compared to a TTS for use in a method of treatment with the same dosing strength but a dosing interval of up to 168 hours or 7 days (an around-the-clock treatment with a once-a-week TTS exchange mode).

In certain embodiments, the huperzine A has a purity of at least 95%, preferably of at least 98% and more preferably of at least 99% as determined by quantitative HPLC. Quantitative HPLC may be performed with Reversed-Phase-HPLC with UV detection.

### Polymers

As indicated above, the TTS according to the present invention comprises at least one polymer in the huperzine A-containing layer, in particular in the huperzine A-containing matrix layer, wherein the at least one polymer is selected from the group consisting of silicone polymers, polyisobutylenes, acrylate polymers and styrene-isoprene-styrene block copolymers. Further details regarding the polymers in the TTS, in particular regarding the at least one polymer in the huperzine A-containing matrix layer, are provided below.

The self-adhesive properties of the huperzine A-containing layer structure are preferably provided by the at least one polymer in the huperzine A-containing matrix layer. Thus, in a preferred embodiment of the invention, the at least one polymer is a pressure sensitive adhesive polymer.

In a preferred embodiment, the at least one polymer is selected from the group consisting of polyisobutylenes, acrylate polymers and styrene-isoprene-styrene block copolymers.

In a more preferred embodiment, the at least one polymer is an acrylate polymer or a styrene-isoprene-styrene block copolymer.

If the at least one polymer is an acrylate polymer, the polymer is preferably an acrylic copolymer comprising a hydroxyl group based on vinyl acetate, 2-ethylhexyl-acrylate and 2-hydroxyethyl-acrylate.

If the at least one polymer is a styrene-isoprene-styrene block copolymer, the block copolymer preferably contains 10 to 25% by weight of styrene, more preferably 15% by weight of styrene.

Thus, in an even more preferred embodiment, the at least one polymer is an acrylate polymer, wherein the acrylate polymer is preferably an acrylic copolymer comprising a hydroxyl group based on vinyl acetate, 2-ethylhexyl-acrylate and 2-hydroxyethyl-acrylate, or a styrene-isoprene-styrene block copolymer, wherein the styrene-isoprene-styrene block copolymer preferably contains 10 to 25% by weight of styrene, more preferably 15% by weight of styrene.

In one particularly preferred embodiment, the at least one polymer is an acrylate polymer, preferably an acrylic copolymer comprising a hydroxyl group based on vinyl acetate, 2-ethylhexyl-acrylate and 2-hydroxyethyl-acrylate.

In another particularly preferred embodiment, the at least one polymer is a styrene-isoprene-styrene block copolymer, preferably a styrene-isoprene-styrene block copolymer which contains 10 to 25% by weight of styrene, more preferably 15% by weight of styrene.

In a preferred embodiment, the at least one polymer is present in an amount of from 65 to 90% by weight, preferably from 70 to 85% by weight, even more preferably from 75 to 80% by weight, based on the total weight of the huperzine A-containing layer.

The TTS of the present invention, in particular the huperzine A-containing matrix layer, may comprise only one polymer or a combination of polymers as defined herein. Additional polymers may also be added to enhance cohesion and/or adhesion. Thus, it is to be understood that the above indicated amounts of the at least one polymer may refer to one polymer only, but also to a combination of polymers as defined herein. However, it has been found that using only one polymer, in particular one polymer as described herein above and below, provides sufficient adhesion of the TTS. Providing a TTS according to the invention comprising a huperzine A-containing matrix layer, which contains only one polymer provides a structure of even lower complexity.

Thus, in a preferred embodiment, the TTS of the present invention, in particular the huperzine A-containing matrix layer, comprises only one polymer. Preferably, the one polymer is selected from the group consisting of polyisobutylenes, acrylate polymers and styrene-isoprene-styrene block copolymers, more preferably from an acrylate polymer or a styrene-isoprene-styrene block copolymer, wherein the acrylate polymer is preferably an acrylic copolymer comprising a hydroxyl group based on vinyl acetate, 2-ethylhexyl-acrylate and 2-hydroxyethyl-acrylate, and wherein the styrene-isoprene-styrene block copolymer preferably contains 10 to 25% by weight of styrene, more preferably 15% by weight of styrene.

In one embodiment, the huperzine A-containing matrix layer comprises:
i) huperzine A; and
ii) at least one, preferably one, polyisobutylene polymer, preferably in an amount of from 65 to 90% by weight, more preferably from 70 to 90% by weight, even more preferably from 75 to 85% by weight, based on the total weight of the huperzine A-containing layer; and
iii) at least one additive selected from crystallization inhibitors, permeation enhancers and combinations thereof.

In a preferred embodiment, the huperzine A-containing matrix layer comprises:
i) huperzine A; and
ii) at least one, preferably one, styrene-isoprene-styrene block copolymer, preferably in an amount of from 65 to 90% by weight, more preferably from 70 to 85% by weight, even more preferably from 70 to 80% by weight, based on the total weight of the huperzine A-containing layer, wherein the styrene-isoprene-styrene block copolymer preferably contains 10 to 25% by weight of styrene, more preferably 15% by weight of styrene; and
iii) at least one additive selected from crystallization inhibitors, permeation enhancers and combinations thereof.

In another preferred embodiment, the huperzine A-containing matrix layer comprises:
i) huperzine A; and
ii) at least one, preferably one, acrylate polymer, preferably in an amount of from 65 to 90% by weight, more preferably from 70 to 85% by weight, even more preferably from 75 to 85% by weight, based on the total weight of the huperzine A-containing layer, wherein the acrylate polymer is preferably an acrylic copolymer comprising a hydroxyl group based on vinyl acetate, 2-ethylhexyl-acrylate and 2-hydroxyethyl-acrylate; and
iii) at least one additive selected from crystallization inhibitors, permeation enhancers and combinations thereof.

### Silicone Polymers

In one embodiment of the present invention, the at least one polymer in the huperzine A-containing matrix layer is a silicone polymer.

As used herein, silicone polymers are polymers based on polysiloxanes. They may therefore also be referred to as silicone-based polymers or polymers based on polysiloxanes. Preferably, the silicone-based polymers are silicone-based pressure sensitive adhesives. Silicone-based pressure sensitive adhesives may also be referred to as silicone pressure sensitive adhesives or pressure-sensitive adhesives based on polysiloxanes.

Suitable silicone-based polymers are commercially available under the brand names BIO-PSAs (pressure sensitive adhesives based on polysiloxanes).

Pressure-sensitive adhesives based on polysiloxanes provide for suitable tack and for quick bonding to various skin types, including wet skin, suitable adhesive and cohesive qualities, long lasting adhesion to the skin, a high degree of flexibility, a permeability to moisture, and compatibility to many actives and film-substrates. It is possible to provide the pressure-sensitive adhesives based on polysiloxanes with sufficient amine resistance and therefore enhanced stability in the presence of amines. Such pressure-sensitive adhesives are based on a resin-in-polymer concept wherein, by condensation reaction of silanol end blocked polydimethylsiloxane with a silica resin (also referred to as silicate resin), a pressure-sensitive adhesive based on polysiloxane is prepared wherein for amine stability the residual silanol functionality is additionally capped with trimethylsiloxy groups. The silanol end blocked polydimethylsiloxane content contributes to the viscous component of the visco-elastic behavior and impacts the wetting and the spreadability properties of the adhesive. The resin acts as a tackifying and reinforcing agent and participates in the elastic component. The correct balance between silanol end blocked polydimethylsiloxane and resin provides for the correct adhesive properties.

In view of the above, silicone-based polymers, and in particular silicone-based pressure sensitive adhesives, are generally obtainable by polycondensation of silanol endblocked polydimethylsiloxane with a silicate resin. Amine-compatible silicone-based polymers, and in particular amine-compatible silicone-based pressure sensitive adhesives, can be obtained by reacting the silicone-based polymer, in particular the silicone-based pressure sensitive adhesive, with trimethylsilyl (e.g. hexamethyldisilazane) in order to reduce the silanol content of the polymer. As a result, the residual silanol functionality is at least partly, preferably mostly or fully capped with trimethylsiloxy groups.

As indicated above, the tackiness of the silicone-based polymer may be modified by the resin-to-polymer ratio, i.e. the ratio of the silanol endblocked polydimethylsiloxane to the silicate resin, which is preferably in the range of from 70:30 to 50:50, preferably from 65:35 to 55:45. The tackiness will be increased with increasing amounts of the polydimethylsiloxane relative to the resin. High tack silicone-based polymers preferably have a resin-to-polymer ratio of 55:45, medium tack silicone-based polymers preferably have a resin-to-polymer ratio of 60:40, and low tack silicone-based polymers preferably have a resin-to-polymer ratio of 65:35. High tack silicone-based polymers preferably have a complex viscosity at 0.01 rad/s and 30° C. of about 5×10⁶ Poise, medium tack silicone-based polymers preferably have a complex viscosity at 0.01 rad/s and 30° C. of about 5×10⁷ Poise, and low tack silicone-based polymers preferably have a complex viscosity at 0.01 rad/s and 30° C. of about 5×10⁸ Poise. High tack amine-compatible silicone-based polymers preferably have a complex viscosity at 0.01 rad/s and 30° C. of about 5×10⁶ Poise, medium tack amine-compatible silicone-based polymers preferably have a complex viscosity at 0.01 rad/s and 30° C. of about 5×10⁸ Poise, and low tack amine-compatible silicone-based polymers preferably have a complex viscosity at 0.01 rad/s and 30° C. of about 5×10⁹ Poise.

Examples of silicone-based PSA compositions which are commercially available include the standard BIO-PSA series (7-4400,7-4500 and 7-4600 series) and the amine compatible (endcapped) BIO-PSA series (7-4100, 7-4200 and 7-4300 series) manufactured and typically supplied in n-heptane or ethyl acetate by DuPont™. For example, BIO-PSA 7-4201 is characterized by a solution viscosity at 25° C. and about 60% solids content in heptane of 450 mPa s and a complex viscosity at 0.01 rad/s at 30° C. of 1×10⁸ Poise. BIO-PSA 7-4301 has a solution viscosity at 25° C. and about 60% solids content in heptane of 500 mPa s and a complex viscosity at 0.01 rad/s at 30° C. of 5×10⁶ Poise. BIO-PSA 7-4202 is characterized by a solution viscosity at 25° C. and about 60% solids content in ethyl acetate of 800 mPa s and a complex viscosity at 0.01 rad/s at 30° C. of 1×10⁸ Poise. BIO-PSA 7-4302 has a solution viscosity at 25° C. and about 60% solids content in ethyl acetate of 1200 mPa s and a complex viscosity at 0.01 rad/s at 30° C. of 5×10⁶ Poise.

The pressure-sensitive adhesives based on polysiloxanes are supplied and used in solvents like n-heptane, ethyl acetate or other volatile silicone fluids. The solids content of pressure-sensitive adhesives based on polysiloxanes in solvents is usually between 60 and 85%, preferably between 70 and 80% or between 60 and 75%. The skilled person is aware that the solids content may be modified by adding a suitable amount of solvent.

Examples of silicone-based PSA compositions which are commercially available include the standard BIO-PSA series (7-4400,7-4500 and 7-4600 series) and the amine compatible (endcapped) BIO-PSA series (7-4100, 7-4200 and 7-4300 series) manufactured and typically supplied in n-heptane or ethyl acetate by DuPont™. For example, BIO-PSA 7-4201 is characterized by a solution viscosity at 25° C. and about 60% solids content in heptane of 450 mPa s and a complex viscosity at 0.01 rad/s at 30° C. of 1×10⁸ Poise.

The preferred pressure-sensitive adhesives based on polysiloxanes in accordance with the invention are characterized by a solution viscosity at 25° C. and 60% solids content in n-heptane of more than about 150 mPa s, or from about 200 mPa s to about 700 mPa s, preferably as measured using a Brookfield RVT viscometer equipped with a spindle number 5 at 50 rpm. These may also be characterized by a complex viscosity at 0.01 rad/s at 30° C. of less than about 1×10⁹ Poise or from about 1×10⁵ to about 9×10⁸ Poise.

### Polyisobutylenes

In one embodiment of the present invention, the at least one polymer in the huperzine A-containing matrix layer is a polyisobutylene.

Preferably, the polyisobutylenes are pressure-sensitive adhesives based on polyisobutylenes. Pressure-sensitive adhesives based on polyisobutylenes may also be referred to as polyisobutylene-based pressure-sensitive adhesives.

In one embodiment, the polyisobutylene refers to a mixture being a combination of low molecular weight polyisobutylene and high molecular weight polyisobutylene in a mass ratio of the mass of the low molecular weight polyisobutylene to the mass of the high molecular weight polyisobutylene ranging from 99:1 to 50:50, and wherein preferably the low molecular weight polyisobutylene has a viscosity average molecular weight of from 38,000 to 42,000 g/mol and/or a weight average molecular weight of from 34,000 to 40,000 g/mol, and wherein the high molecular weight polyisobutylene has a viscosity average molecular weight of from 1,100,000 to 1,120,000 g/mol and/or a weight average molecular weight of from 1,540,000 to 1,560,000 g/mol.

Suitable polyisobutylenes according to the invention, in particular pressure-sensitive adhesives based on polyisobutylenes, are available under the tradename Oppanol®. Combinations of high-molecular weight polyisobutylenes (B100/B80) and low-molecular weight polyisobutylenes (B10, B11, B12, B13) may be used. Suitable ratios of low-molecular weight polyisobutylene to high-molecular weight polyisobutylene are in the range of from 100:1 to 1:100, preferably from 95:5 to 40:60, more preferably from 90:10 to 80:20. A preferred example for a polyisobutylene combination is B10/B100 in a ratio of 85/15. Oppanol® B100 has a viscosity average molecular weight Mᵥ of 1,110,000, and a weight average molecular weight M_{w} of 1,550,000, and an average molecular weight distribution M_{w}/Mₙ of 2.9. Oppanol® B10 has a viscosity average molecular weight Mᵥ of 40,000, and a weight average molecular weight M_{w} of 53,000, and an average molecular weight distribution M_{w}/Mₙ of 3.2.

Other suitable polyisobutylenes according to the invention, in particular pressure-sensitive adhesives based on polyisobutylenes, are available from Henkel under the tradename Duro-Tak®. In certain embodiments, polybutene may be added to the polyisobutylenes.

In certain embodiments, the polyisobutylene is accessible by blending of 50 to 95%, preferably of 60 to 90%, and in particular of 75 to 90% by weight, based on the total weight of the polyisobutylene with 5 to 50%, preferably 10 to 40%, and in particular 10 to 25% by weight, based on the total weight of the polybutene. In this connection, it is preferred that the weight ratio of the mass of the polyisobutylene to the mass of the polybutene is of 20:1 to 1:1 and in particular of 8:1 to 3:1.

Polyisobutylenes, in particular pressure-sensitive adhesives based on polyisobutylenes, are usually supplied and used in solvents, such as e.g. n-heptane and ethyl acetate. The solids content of polyisobutylenes in solvents is usually between 30 and 50%, preferably between 35 and 40%. The skilled person is aware that the solids content may be modified by adding a suitable amount of solvent.

In a preferred embodiment according to the invention, the polyisobutylene is DURO-TAK 87-6908 (having about 85% by weight of polyisobutylene and 15% by weight of polybutene, provided in n-heptane) provided by Henkel.

Thus, in one embodiment, the huperzine A-containing matrix layer comprises:
i) huperzine A; and
ii) at least one, preferably one, polyisobutylene polymer, preferably in an amount of from 65 to 90% by weight, more preferably from 70 to 90% by weight, even more preferably from 75 to 85% by weight, based on the total weight of the huperzine A-containing layer, wherein the polyisobutylene polymer is provided as a solution with a solids content of between 30 and 50%, preferably between 35 and 40%; and
iii) at least one additive selected from crystallization inhibitors, permeation enhancers and combinations thereof.

### Styrene-Isoprene-Styrene Block Copolymers

In another embodiment of the present invention, the at least one polymer in the huperzine A-containing matrix layer is a styrene-isoprene-styrene block copolymer. In particular, the styrene-isoprene-styrene block copolymer is a thermoplastic elastomer.

In a preferred embodiment, the styrene-isoprene-styrene block copolymer contains 10 to 25% by weight of styrene. Accordingly, styrene-isoprene-styrene block copolymer preferably contains 75 to 90% of isoprene.

It is preferred that the mass of the styrene to the mass of the isoprene is of 1:20 to 1:2, preferably of 1:15 to 1:3.

The thermoplastic styrene-isoprene-styrene block copolymers can also be characterized in view of their melt flow ratio (MFR), i.e., the case of flow of the melt.

Preferably, the styrene-isoprene block copolymers have a melt flow rate (determined according to JIS K7210, 200° C., 49.0 N) of 1 to 30 g/10 min, preferably of 5 to 20 g/10 min, and in particular of 6 to 15 g/10 min.

Suitable styrene-isoprene-styrene block copolymers according to the invention are available from JSR Corporation. Specific styrene-isoprene-styrene block copolymers are available as:
JSR SIS5002 (a block copolymer containing 22% by weight of styrene and 78% by weight of isoprene, MFR 7 g/10 min),
JSR SIS5229 (a block copolymer containing 15% by weight of styrene and 85% by weight of isoprene, MFR 11 g/10 min), 1 JSR SIS5250 (a block copolymer containing 20% by weight of styrene and 80% by weight of isoprene, MFR 8.5 g/10 min),
JSR SIS5405 (a block copolymer containing 15% by weight of styrene and 85% by weight of isoprene, MFR 20 g/10 min),
JSR SIS5505 (a block copolymer containing 16% by weight of styrene and 84% by weight of isoprene, MFR 8 g/10 min).

In a particularly preferred embodiment, the styrene-isoprene-styrene block copolymer contains 15% by weight of styrene. Accordingly, in a particularly preferred embodiment, the styrene-isoprene-styrene block copolymer contains 85% of isoprene.

Thus, particularly preferred styrene-isoprene-styrene block copolymers according to the invention are JSR SIS5229 (a block copolymer containing 15% by weight of styrene and 85% by weight of isoprene, MFR 11 g/10 min) and JSR SIS5405 (a block copolymer containing 15% by weight of styrene and 85% by weight of isoprene, MFR 20 g/10 min), even more preferably JSR SIS5229 (a block copolymer containing 15% by weight of styrene and 85% by weight of isoprene, MFR 11 g/10 min).

Styrene-isoprene-styrene block copolymers are usually supplied as particles.

In certain embodiments, the styrene-isoprene-styrene block copolymers as described above are used together with a tackifier resin. Preferably, the tackifier is a thermoplastic ester resin, and in particular a thermoplastic ester resin derived from glycerol and a highly stabilized rosin. Preferably, the tackifier has an acid number (mg KOH/g; determined according to ASTM D465) of 3 to 12, preferably of 4 to 10, and in particular of 5 to 9.

According to the present invention, the term “rosin” refers to a natural product obtained from the exudation of trees of the family Pinus. Rosin consists of about 90% by weight or more based on the total weight of the rosin, of mixed unsaturated acids. The rosin acids are mainly monobasic carboxylic acids, all containing the penanthrene skeleton and all having 20 carbon atoms in the molecule, the main difference between the acids being the number and position of the double bonds. The acids can have conjugated double bonds like in abietic acid or a non-conjugated double bond as in pimaric acid. Natural rosin may be classified in several types due to its origin. There are, gum rosin, wood rosin and tall oil rosin (for further information see e.g. “Modern Surface Coatings” Paul Nylen et al, Interscience Publishers 1965, p 133-137). Because of its structure, rosin is generally unsuited for use in adhesive formulations. The double bonds of the rosin acids result in instability against oxidation and the isomer composition causes the rosin to crystallize. Therefore, rosin must be subjected to chemical reactions altering the double bond position and changing the rosin acid isomer composition. Processes for stabilizing rosin are well known (stabilizing by hydrogenation, dehydrogenation or disproportionation).

In a preferred embodiment, the ratio of styrene-isoprene-styrene block copolymer to tackifier resin is from 1:1 to 1:5, preferably from 1:2 to 1:4, more preferably 1:3.

Thus, in one embodiment, the TTS according to the invention, in particular the huperzine A-containing matrix layer comprises:
i) huperzine A; and
ii) at least one, preferably one, styrene-isoprene-styrene block copolymer, preferably in an amount of from 65 to 90% by weight, more preferably from 70 to 85% by weight, even more preferably from 70 to 80% by weight, based on the total weight of the huperzine A-containing layer, wherein the styrene-isoprene-styrene block copolymer preferably contains 10 to 25% by weight of styrene, more preferably 15% by weight of styrene, and wherein the styrene-isoprene-styrene block copolymer preferably contains a tackifier resin in a ratio of styrene-isoprene-styrene block copolymer to tackifier resin from 1:1 to 1:5, preferably from 1:2 to 1:4, more preferably 1:3; and
iii) at least one additive selected from crystallization inhibitors, permeation enhancers and combinations thereof.

### Acrylate Polymers

In another embodiment of the present invention, the at least one polymer in the huperzine A-containing matrix layer is an acrylate polymer.

As used herein, the terms acrylic polymer and acrylate polymer are synonymously used. Preferably, the acrylic polymers are pressure-sensitive adhesives based on acrylates. Pressure-sensitive adhesives based on acrylates may also be referred to as acrylate-based pressure-sensitive adhesives, or acrylate pressure-sensitive adhesives.

Acrylate polymers, in particular pressure-sensitive adhesives based on acrylates, may be provided in the form of a solution with a solids content preferably between 30% and 60%.

Acrylate polymers, in particular acrylate-based pressure-sensitive adhesives, may or may not comprise functional groups such as hydroxy groups, carboxylic acid groups, neutralized carboxylic acid groups and mixtures thereof. Thus, the term “functional groups” in particular refers to hydroxy- and carboxylic acid groups, and deprotonated carboxylic acid groups.

Corresponding commercial products are available e.g. from Henkel under the tradename Duro-Tak®. Such acrylate polymers, in particular acrylate-based pressure-sensitive adhesives, are based on monomers selected from one or more of acrylic acid, butylacrylate, 2-ethylhexylacrylate, glycidylmethacrylate, 2-hydroxyethylacrylate, methylacrylate, methylmethacrylate, butylmethacrylate, t-octylacrylamide and vinylacetate, and are provided in ethyl acetate, heptane, n-heptane, hexane, methanol, ethanol, isopropanol, 2,4-pentanedione, toluene or xylene or mixtures thereof.

Specific acrylate polymers, in particular acrylate-based pressure-sensitive adhesives, are available as:
Duro-Tak™ 387-2287 or Duro-Tak™ 87-2287 (a copolymer based on vinyl acetate, 2-ethylhexyl-acrylate, 2-hydroxyethyl-acrylate and glycidyl-methacrylate provided as a solution in ethyl acetate without cross-linking agent),
Duro-Tak™ 387-2516 or Duro-Tak™ 87-2516 (a copolymer based on vinyl acetate, 2-ethylhexyl-acrylate, 2-hydroxyethyl-acrylate and glycidyl-methacrylate provided as a solution in ethyl acetate, ethanol, n-heptane, and methanol with a titanium cross-linking agent),
Duro-Tak™ 387-2051 or Duro-Tak™ 87-2051 (a copolymer based on acrylic acid, butylacrylate, 2-ethylhexylacrylate and vinyl acetate, provided as a solution in ethyl acetate or heptane without cross-linking agent),
Duro-Tak™ 387-2353 or Duro-Tak™ 87-2353 (a copolymer based on acrylic acid, 2-ethylhexylacrylate, glycidylmethacrylate and methylacrylate, provided as a solution in ethyl acetate and hexane),
Duro-Tak™ 87-4098 (a copolymer based on 2-ethylhexyl-acrylate and vinyl acetate, provided as a solution in ethyl acetate).
Duro-Tak™ 87-4287 (a copolymer comprising a hydroxyl group based on vinyl acetate, 2-ethylhexyl-acrylate and 2-hydroxyethyl-acrylate, provided as a solution in ethyl acetate without cross-linking agent).
Duro-Tak™ 387-2054 or Duro-Tak™ 87-2054 (a copolymer based on acrylic acid, butylacrylate, 2-ethylhexylacrylate and vinyl acetate, provided as a solution in ethyl acetate or heptane with cross-linking agent).
Duro-Tak™ 87-9301 (an acrylic copolymer comprising no functional groups, obtained without using a crosslinking agent, provided in ethyl acetate),
Duro-Tak™ 387-2510 or Duro-Tak™ 87-2510 (a copolymer based on 2-ethylhexyl-acrylate, 2-hydroxyehtly-acrylate and methylacrylate, provided as a solution in ethyl acetate and hexane).

Preferred acrylate polymers, in particular acrylate-based pressure sensitive adhesives, according to the present invention are Duro-Tak™ 87-4287 (a copolymer comprising a hydroxyl group based on vinyl acetate, 2-ethylhexyl-acrylate and 2-hydroxyethyl-acrylate, provided as a solution in ethyl acetate without cross-linking agent) and Duro-Tak™ 87-9301 (an acrylic copolymer comprising no functional groups, obtained without using a crosslinking agent, provided in ethyl acetate). Particularly preferred is Duro-Tak™ 87-4287 (a copolymer comprising a hydroxyl group based on vinyl acetate, 2-ethylhexyl-acrylate and 2-hydroxyethyl-acrylate, provided as a solution in ethyl acetate without cross-linking agent). It has been found by the inventors that using a copolymer comprising a hydroxyl group based on vinyl acetate, 2-ethylhexyl-acrylate and 2-hydroxyethyl-acrylate, provided as a solution in ethyl acetate, such as Duro-Tak™ 87-4287, provides a TTS with advantageous skin permeation properties such as advantageous skin flux and cumulated permeation amount.

As indicated above, acrylate polymers are usually supplied and used in solvents, such as e.g. n-heptane and ethyl acetate. The solids content of acrylate polymers in solvents is preferably between 25 and 60%, more preferably between 30 and 50%, even more preferably between 35 and 40%. The skilled person is aware that the solids content may be modified by adding a suitable amount of solvent.

Thus, in one preferred embodiment, the TTS according to the invention, in particular the huperzine A-containing matrix layer, comprises:
i) huperzine A; and
ii) at least one, preferably one, acrylate polymer, preferably in an amount of from 65 to 90% by weight, more preferably from 70 to 85% by weight, even more preferably from 75 to 85% by weight, based on the total weight of the huperzine A-containing layer, wherein the acrylate polymer is preferably an acrylic copolymer comprising a hydroxyl group based on vinyl acetate, 2-ethylhexyl-acrylate and 2-hydroxyethyl-acrylate, and wherein the solids content of the acrylate polymer is preferably between 25 and 60%, more preferably between 30 and 50%, even more preferably between 35 and 40%; and
iii) at least one additive selected from crystallization inhibitors, permeation enhancers and combinations thereof.

### Additives

As indicated above, the TTS according to the invention, and in particular the huperzine A-containing matrix layer, comprises at least one additive selected from crystallization inhibitors, permeation enhancers and combinations thereof.

In one embodiment, the at least one additive is a permeation enhancer. By adding a permeation enhancer, the skin flux and permeation can be increased. Thus, in a preferred embodiment, the huperzine A-containing layer comprises at least one, preferably one or two, permeation enhancer.

In another embodiment, the at least one additive is a crystallization inhibitor. By adding a crystallization enhancer, the solubility of huperzine A in the huperzine A-containing matrix layer, in particular in the at least one polymer in the huperzine A-containing matrix layer can be increased. Thus, in a preferred embodiment, the huperzine A-containing layer comprises at least one, preferably one or two, more preferably one, crystallization inhibitor.

In certain embodiments, combinations of the afore-mentioned additives are preferred. Thus, in preferred embodiments, the huperzine A-containing matrix layer comprises at least two additives, wherein the first additive is a crystallization inhibitor and the second additive is a permeation inhibitor. In particular, in preferred embodiments, the huperzine A-containing layer comprises at least one, preferably one or two, permeation enhancers and at least one, preferably one, crystallization inhibitor.

In view of the above, the TTS according to the invention, and in particular the huperzine A-containing matrix layer, comprises at least one, preferably from 1 to 5, more preferably from 1 to 4, even more preferably one, two, or three, additive selected from crystallization inhibitors, permeation enhancers and combinations thereof.

In a particularly preferred embodiment, the TTS according to the invention, and in particular the huperzine A-containing matrix layer, comprises at least one, preferably one additive selected from crystallization inhibitors, permeation enhancers and combinations thereof.

In another particularly preferred embodiment, the TTS according to the invention, and in particular the huperzine A-containing matrix layer, comprises at least two, preferably two, additives selected from crystallization inhibitors, permeation enhancers and combinations thereof.

In another particularly preferred embodiment, the TTS according to the invention, and in particular the huperzine A-containing matrix layer, comprises at least three, preferably three, additives selected from crystallization inhibitors, permeation enhancers and combinations thereof.

In one particularly preferred embodiment, the huperzine A-containing matrix layer comprises one crystallization inhibitor and one permeation enhancer. In another preferred embodiment, the huperzine A-containing matrix layer comprises one crystallization inhibitor and two permeation enhancers.

Thus, in one embodiment, the TTS according to the invention, in particular the huperzine A-containing matrix layer, comprises:
i) huperzine A; and
ii) at least one, preferably one, polymer as described above; and
iii) at least two additives, wherein the additives are at least one, preferably one or two, permeation enhancers and at least one, preferably one, crystallization inhibitor.

In one particular embodiment, the TTS according to the invention, in particular the huperzine A-containing matrix layer, comprises:
i) huperzine A; and
ii) at least one, preferably one, polymer as described above; and
iii) two additives, wherein the additives are one permeation enhancer and one crystallization inhibitor.

In another particular embodiment, the TTS according to the invention, in particular the huperzine A-containing matrix layer, comprises:
i) huperzine A; and
ii) at least one, preferably one, polymer as described above; and
iii) three additives, wherein the additives are two permeation enhancers and one crystallization inhibitor.

Further details regarding these additives, in particular the at least one crystallization inhibitor and/or at least one permeation enhancer are provided below.

### Crystallization Inhibitors

In certain preferred embodiments, the TTS according to the invention, in particular the huperzine A-containing matrix layer comprises a crystallization inhibitor. Accordingly, in a preferred embodiment, the at least one additive in the huperzine A-containing matrix layer comprises a crystallization inhibitor. By adding a crystallization enhancer, the solubility of huperzine A in the huperzine A-containing matrix layer, in particular in the at least one polymer in the huperzine A-containing matrix layer can be increased.

Thus, in a preferred embodiment, the huperzine A-containing layer comprises at least one, preferably one or two, more preferably one, crystallization inhibitor.

If the huperzine A-containing layer comprises at least one may or may not comprise comprises at least one, preferably one or two, more preferably one, crystallization inhibitor, the huperzine A-containing layer may or may not comprise in addition one or more further additives. For example, one or more permeation enhancers may be added to increase the skin flux and/or cumulative permeation of huperzine A.

Thus, in a preferred embodiment, the huperzine A-containing layer comprises at least one, preferably one or two, permeation enhancers and at least one, preferably one or two, more preferably one, crystallization inhibitor.

Suitable examples of crystallization inhibitors include polyvinylpyrrolidone, vinyl acetate/vinylpyrrolidone copolymer and cellulose derivatives. The crystallization inhibitor is preferably polyvinylpyrrolidone, in particular soluble polyvinylpyrrolidone or insoluble/cross-linked polyvinylpyrrolidones.

The term “soluble polyvinylpyrrolidone” refers to polyvinylpyrrolidone, also known as povidone or PVP, which is soluble with more than 10% in at least ethanol, preferably also in water, diethylene glycol, methanol, n-propanol, 2 propanol, n-butanol, chloroform, methylene chloride, 2-pyrrolidone, macrogol 400, 1,2 propylene glycol, 1,4 butanediol, glycerol, triethanolamine, propionic acid and acetic acid. Examples of polyvinylpyrrolidones which are commercially available include Kollidon® 12 PF, Kollidon® 17 PF, Kollidon® 25, Kollidon® 30 and Kollidon® 90 F supplied by BASF, or povidone K90F. The different grades of Kollidon® are defined in terms of the K-Value reflecting the average molecular weight of the polyvinylpyrrolidone grades. Kollidon® 12 PF is characterized by a K-Value range of 10.2 to 13.8, corresponding to a nominal K-Value of 12. Kollidon® 17 PF is characterized by a K-Value range of 15.3 to 18.4, corresponding to a nominal K-Value of 17. Kollidon® 25 is characterized by a K-Value range of 22.5 to 27.0, corresponding to a nominal K-Value of 25, Kollidon® 30 is characterized by a K-Value range of 27.0 to 32.4, corresponding to a nominal K-Value of 30. Kollidon® 90 F is characterized by a K-Value range of 81.0 to 97.2, corresponding to a nominal K-Value of 90. Preferred Kollidon® grades are Kollidon® 12 PF, Kollidon® 30 and Kollidon® 90 F. Within the meaning of this invention, the term “K-Value” refers to a value calculated from the relative viscosity of polyvinylpyrrolidone in water according to the European Pharmacopoeia (Ph.Eur.) and USP monographs for “Povidone”.

The term “insoluble/cross-linked polyvinylpyrrolidones” refers to cross-linked derivatives of polyvinylpyrrolidones such as the highly cross-linked modification polyvinylpolypyrrolidone, also known as crospovidone or PVPP.

In a preferred embodiment of the invention, the crystallization inhibitor is selected from polyvinylpyrrolidone (PVP, povidone), polyvinylpolypyrrolidone (PVPP, crospovidone) and combinations thereof.

In one particularly preferred embodiment, the crystallization inhibitor is polyvinylpyrrolidone (PVP, povidone). In another particularly preferred embodiment, the crystallization inhibitor is polyvinylpolypyrrolidone (PVPP, crospovidone).

If the huperzine A-containing layer comprises at least one, preferably one or two, more preferably one, crystallization inhibitor, the huperzine A-containing layer may or may not comprise in addition one or more further additives. For example, one or more permeation enhancers may be added to increase the skin flux and/or cumulative permeated amount of huperzine A.

Thus, in a preferred embodiment, the huperzine A-containing layer of the TTS according to the invention or any of the preferred embodiments defined above, comprises at least one, preferably one or two, more preferably one, crystallization inhibitor and at least one, preferably one or two, permeation enhancers.

In one embodiment, the TTS according to the invention, in particular the huperzine A-containing matrix layer, comprises:
i) huperzine A; and
ii) at least one, preferably one, polymer as described above; and
iii) at least two additives, wherein the first additive is at least one, preferably one or two, permeation enhancers and the second additive is at least one, preferably one, crystallization inhibitor, selected from polyvinylpyrrolidone (PVP, povidone) and polyvinylpolypyrrolidone (PVPP, crospovidone).

In one particular embodiment, the TTS according to the invention, in particular the huperzine A-containing matrix layer, comprises:
i) huperzine A; and
ii) at least one, preferably one, polymer as described above; and
iii) one or two permeation enhancer and one crystallization inhibitor, wherein the crystallization inhibitor is polyvinylpyrrolidone (PVP, povidone).

In another particular embodiment, the TTS according to the invention, in particular the huperzine A-containing matrix layer, comprises:
i) huperzine A; and
ii) at least one, preferably one, polymer as described above; and
iii) one or two permeation enhancer and one crystallization inhibitor, wherein the crystallization inhibitor is polyvinylpolypyrrolidone (PVPP, crospovidone).

In particular, if the at least one polymer in the huperzine A-containing layer is an acrylate polymer, the crystallization inhibitor is preferably polyvinylpyrrolidone (PVP, povidone).

Thus, in one embodiment, the huperzine A-containing layer comprises:
i) huperzine A; and
ii) at least one, preferably one, acrylate polymer, preferably in an amount of from 65 to 90% by weight, more preferably from 70 to 85% by weight, even more preferably from 75 to 85% by weight, based on the total weight of the huperzine A-containing layer, wherein the acrylate polymer is preferably an acrylic copolymer comprising a hydroxyl group based on vinyl acetate, 2-ethylhexyl-acrylate, and 2-hydroxyethyl-acrylate; and
iii) at least one, preferably one, crystallization inhibitor selected from polyvinylpyrrolidone (PVP, povidone) and optionally a second additive, wherein the second additive is preferably a permeation enhancer.

If the at least one polymer in the huperzine A-containing layer is a polyisobutylene or a styrene-isoprene-styrene block copolymer, the crystallization inhibitor is preferably polyvinylpolypyrrolidone (PVPP, crospovidone).

Thus, in another embodiment, the huperzine A-containing layer comprises:
i) huperzine A; and
ii) at least one, preferably one, polyisobutylene, preferably in an amount of from 65 to 90% by weight, more preferably from 70 to 90% by weight, even more preferably from 75 to 85% by weight, based on the total weight of the huperzine A-containing layer, wherein the polyisobutylene is preferably provided as a solution with a solids content of between 30 and 50%, preferably between 35 and 40%, and
iii) at least one, preferably one, crystallization inhibitor selected from polyvinylpolypyrrolidone (PVPP, crospovidone) and optionally a second additive, wherein the second additive is preferably a permeation enhancer.

In another embodiment, the huperzine A-containing layer comprises:
i) huperzine A; and
ii) at least one, preferably one, styrene-isoprene-styrene block copolymer, preferably in an amount of from 65 to 90% by weight, more preferably from 70 to 85% by weight, even more preferably from 70 to 80% by weight, based on the total weight of the huperzine A-containing layer, wherein the styrene-isoprene-styrene block copolymer preferably contains 10 to 25% by weight of styrene, more preferably 15% by weight of styrene, and
iii) at least one, preferably one, crystallization inhibitor selected from polyvinylpolypyrrolidone (PVPP, crospovidone) and optionally a second additive, wherein the second additive is preferably a permeation enhancer.

In connection with the crystallization inhibitor in the huperzine A-containing matrix layer according to the invention, in particular the embodiments as described above, the crystallization inhibitor is preferably present in an amount of from 5 to 25% by weight, preferably from 5 to 15% by weight or from 15 to 25% by weight, more preferably from 8 to 12% by weight or from 18 to 22% by weight, based on the total weight of the huperzine A-containing layer.

### Permeation Enhancers

In connection with the TTS according to the present invention, in particular the preferred embodiments as described above, the following applies regarding the permeation enhancers. As indicated above, in certain preferred embodiments, the huperzine A-containing layer comprises a permeation enhancer.

Permeation enhancers are substances, which influence the barrier properties of the stratum corneum in the sense of increasing the active agent permeability. Some examples of permeation enhancers are polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; urea and urea derivatives such as allantoin; polar solvents such as dimethyldecylphosphoxide, methylcetylsulfoxide, dimethylaurylamine, dodecyl pyrrolidone, isosorbitol, dimethylacetonide, dimethylsulfoxide, decylmethylsulfoxide, and dimethylformamide; salicylic acid; transcutol, lauryl lactate, methyl laurate, capryol 90, 1-dodecanol, levulinic acid, amino acids; benzyl nicotinate; and higher molecular weight aliphatic surfactants such as lauryl sulfate salts. Other agents include oleic and linoleic acids, ascorbic acid, panthenol, butylated hydroxytoluene, tocopherol, tocopheryl acetate, tocopheryl linoleate, propyl oleate, and isopropyl palmitate.

By adding a permeation enhancer, the skin flux and permeation can be increased. Thus, in the context of the present invention, it is preferred that the huperzine A-containing matrix layer comprises at least one permeation enhancer. In certain preferred embodiments, the TTS of the present invention, in particular the huperzine A-containing matrix layer comprises at least one permeation enhancer, preferably one, two, or three, even more preferably one or two permeation enhancers. In a particularly preferred embodiment, the TTS of the present invention, in particular the huperzine A-containing matrix layer comprises one permeation enhancer. In another particularly preferred embodiment, the TTS of the present invention, in particular the huperzine A-containing matrix layer comprises two permeation enhancers. The latter embodiment refers to the permeation enhancer being a combination of two permeation enhancers.

In a preferred embodiment of the present invention, the permeation enhancer is selected from the group consisting of transcutol, lauryl lactate, methyl laurate, capryol 90, 1-dodecanol, levulinic acid and combinations thereof.

In a more preferred embodiment, the permeation enhancer is selected from methyl laurate, capryol 90, 1-dodecanol and combinations thereof.

It has surprisingly been found by the inventors of the present invention that a TTS comprising methyl laurate, 1-dodecanol, or a combination thereof as permeation enhancer shows an enhanced skin permeation rate and thus cumulative permeated amount of huperzine A. In particular, it has surprisingly been found by the inventors of the present invention that a TTS comprising methyl laurate, 1-dodecanol or combinations thereof in the huperzine A-containing layer shows an enhanced cumulative permeated amount of huperzine A over at least 72 h in comparison to a TTS comprising other permeation enhancers in the huperzine A-containing layer. Further, it has surprisingly been found that the skin permeation rate and thus the cumulative permeated amount of huperzine A can be particularly increased upon using methyl laurate as permeation enhancer and even more increased upon using a combination of methyl laurate and 1-dodecanol as permeation enhancer.

Thus, in a preferred embodiment, the TTS according to the present invention, in particular the huperzine A-containing matrix layer comprises a permeation enhancer, wherein the permeation enhancer is selected from methyl laurate, 1-dodecanol, or combinations thereof, preferably methyl laurate or a combination of methyl laurate and 1-dodecanol.

In a particularly preferred embodiment, the permeation enhancer is methyl laurate.

In another particularly preferred embodiment, the permeation enhancer is a combination of methyl laurate and 1-dodecanol. If a combination of methyl laurate and 1-dodecanol is used, preferably the ratio of methyl laurate:1-dodecanol is from 1:3 to 3:1, more preferably from 1:2 to 2:1, even more preferably from 1:1.5 to 1.5:1, particularly preferably around 1:1.

The skilled person knows how to select the permeation enhancer depending on the desired dosing interval. For example, if the TTS is for use in a method of treatment with a dosing interval of 24 hours or 1 day (an around-the-clock treatment with a once-a-day TTS exchange mode) an increased permeation rate is advantageous. Therefore, a TTS comprising methyl laurate and 1-dodecanol as permeation enhancers is particularly suitable for a TTS for use in a method of treatment with a dosing interval of 24 hours or 1 day. A similar TTS comprising only methyl laurate as permeation enhancer may be particularly suitable for a dosing interval of up to 84 hours or 3.5 days (an around-the clock treatment with a twice-a-week TTS exchange mode) or a dosing interval of up to 168 hours or 7 days (an around-the-clock treatment with a once-a-week TTS exchange mode).

In connection with the permeation enhancer in the huperzine A-containing matrix layer according to the invention, the permeation enhancer is preferably present in an amount of from 2 to 15% by weight, preferably from 2.5 to 7.5% by weight or from 7.5 to 12.5% by weight, based on the total weight of the huperzine A-containing layer. In a particularly preferred embodiment, the permeation enhancer is present in an amount of from 2.5 to 7.5% by weight, based on the total weight of the huperzine A-containing layer.

The TTS of the present invention, in particular the huperzine A-containing matrix layer may comprise only one permeation enhancer or a combination of permeation enhancers as defined herein. It is to be understood that the above indicated amounts of the permeation enhancer may refer to one polymer only, but also to a combination of permeation enhancers as defined herein. If a combination of permeation enhancers is present, the amount of all permeation enhancers together is preferably from 7.5 to 12.5% by weight, based on the total weight of the huperzine A-containing layer. If one permeation enhancer is present, the permeation enhancer is preferably present in an amount of from 2.5 to 7.5% by weight, based on the total weight of the huperzine A-containing layer.

If the huperzine A-containing layer comprises at least one, preferably one or two, permeation enhancers, the huperzine A-containing layer may or may not comprise in addition one or more further additives. For example, one or more, preferably one, crystallization inhibitor as described above may be added to increase the dissolution of huperzine A in the huperzine A-containing matrix layer.

Thus, in preferred embodiments, the huperzine A-containing matrix layer comprises at least two additives, wherein the first additive is a crystallization inhibitor and the second additive is a permeation inhibitor. In particular, in certain preferred embodiments, the huperzine A-containing layer comprises at least one, preferably one or two, permeation enhancers and at least one, preferably one, crystallization inhibitor.

In one embodiment, the TTS according to the invention, in particular the huperzine A-containing matrix layer, comprises:
i) huperzine A; and
ii) at least one, preferably one, polymer as described above; and
iii) at least one, preferably one, two, or three, additive, wherein the additive is at least one, preferably one or two, permeation enhancers, selected from methyl laurate, 1-dodecanol, or a combination thereof, preferably methyl laurate or a combination of methyl laurate and 1-dodecanol, and optionally a second additive, wherein the second additive is preferably at least one, preferably one, crystallization inhibitor, preferably selected from polyvinylpyrrolidone (PVP, povidone) and polyvinylpolypyrrolidone (PVPP, crospovidone).

In a preferred embodiment, the TTS according to the invention, in particular the huperzine A-containing matrix layer, comprises:
i) huperzine A; and
ii) at least one, preferably one, polymer as described above; and
iii) at least two, preferably two or three, additives, wherein the first additive is at least one, preferably one or two, permeation enhancers, selected from methyl laurate, 1-dodecanol, or a combination thereof, preferably methyl laurate or a combination of methyl laurate and 1-dodecanol, and the second additive is at least one, preferably one, crystallization inhibitor, preferably selected from polyvinylpyrrolidone (PVP, povidone) and polyvinylpolypyrrolidone (PVPP, crospovidone).

In one particular embodiment, the TTS according to the invention, in particular the huperzine A-containing matrix layer, comprises:
i) huperzine A; and
ii) at least one, preferably one, polymer as described above; and
iii) at least two, preferably two, additives, wherein the first additive is one permeation enhancer, wherein the permeation enhancer is methyl laurate, and the second additive is preferably one crystallization inhibitor, wherein the crystallization inhibitor is preferably selected from polyvinylpyrrolidone (PVP, povidone) and polyvinylpolypyrrolidone (PVPP, crospovidone).

In another particular embodiment, the TTS according to the invention, in particular the huperzine A-containing matrix layer, comprises:
i) huperzine A; and
ii) at least one, preferably one, polymer as described above; and
iii) at least two additives, wherein the first additive is a permeation enhancer, wherein the permeation enhancers is a combination of methyl laurate and 1-dodecanol, preferably in a ratio of methyl laurate:1-dodecanol from 1:3 to 3:1, more preferably from 1:2 to 2:1, even more preferably from 1:1.5 to 1.5:1, particularly preferably around 1:1, and the second additive is preferably one crystallization inhibitor, wherein the crystallization inhibitor is selected from polyvinylpyrrolidone (PVP, povidone) and polyvinylpolypyrrolidone (PVPP, crospovidone).

If a combination of two permeation enhancers and one crystallization inhibitor is present in the huperzine A-containing matrix layer, as, e.g., in the embodiment described above, this may also be regarded as a huperzine A-containing layer comprising three additives, namely two permeation enhancers and one crystallization inhibitor.

Thus, in another particular embodiment, the TTS according to the invention, in particular the huperzine A-containing matrix layer, comprises:
i) huperzine A; and
ii) at least one, preferably one, polymer as described above; and
iii) at least three, preferably three, additives, wherein the first and second additive is a permeation enhancer, wherein the first permeation enhancers is methyl laurate and the second permeation enhancer is 1-dodecanol, preferably in a ratio of methyl laurate:1-dodecanol from 1:3 to 3:1, more preferably from 1:2 to 2:1, even more preferably from 1:1.5 to 1.5:1, particularly preferably around 1:1, and the third additive is preferably one crystallization inhibitor, wherein the crystallization inhibitor is selected from polyvinylpyrrolidone (PVP, povidone) and polyvinylpolypyrrolidone (PVPP, crospovidone).

### Further Additives

The TTS according to the invention, in particular the huperzine A-containing matrix layer, may comprise, in addition to the at least one crystallization inhibitor and/or permeation enhancer as outlined above, further additives or excipients.

Said further additives or excipients are preferably selected from the group consisting of dispersing agents, solubilizers, film-forming agents, softeners/plasticizers, tackifiers, substances for skincare, pH regulators, preservatives, stabilizing agents, and fillers. Such additives may be present in the huperzine A-containing layer in an amount of from 0.001% to 15% by weight, e.g. from 0.5 to 10% by weight or from 1 to 10% by weight or from 0.01 to 6% by weight, based on the total weight of the huperzine A-containing layer, and wherein the weight % amounts refer to a single additive.

It has been found that the TTS according to the invention provides sufficient permeability of the huperzine A even if no further additives or excipients in addition to the at least one crystallization inhibitor and/or permeation enhancer is present. Thus, in a preferred embodiment, the TTS according to the invention, in particular the huperzine A-containing matrix layer, does not comprise further additives or excipients in addition to the at least one crystallization inhibitor and/or permeation enhancer. Thus, the TTS according to the invention provides a structure of low complexity.

It should be noted that in pharmaceutical formulations, the formulation components are categorized according to their physicochemical and physiological properties, and in accordance with their function. This means in particular that a substance or a compound falling into one category is not excluded from falling into another category of formulation component. E.g. a certain polymer can be a film-forming agent, but also a tackifier. Some substances may e.g. be a typical softener but at the same time act as a permeation enhancer. The skilled person is able to determine based on his general knowledge in which category or categories of formulation components a certain substance or compound belongs to. In the following, details on the excipients and additives are provided which are, however, not to be understood as being exclusive. Other substances not explicitly listed in the present description may be as well used in accordance with the present invention, and substances and/or compounds explicitly listed for one category of formulation component are not excluded from being used as another formulation component in the sense of the present invention.

In one embodiment, the guanfacine-containing layer comprises a dispersing agent. Suitable dispersing agents include esters of fatty acids with polyols, fatty alcohols, polyethylene glycols having an average molecular weight of from 300 to 400, polyethylene glycol alkyl ethers, or silicone polyethers. The dispersing agent is helpful in order to homogeneously disperse the guanfacine within the huperzine A-containing matrix layer, thereby improving the release properties of the TTS.

In one embodiment, the huperzine A-containing matrix layer comprises a solubilizer. The solubilizer preferably improves the dispersibility of the guanfacine in the guanfacine-containing layer and stabilizes the guanfacine-containing layer. Furthermore, the solubilizer may positively influence cohesion. The solubilizer is preferably selected from polyhydric alcohols such as 1,2-propanediol, various butanediols, glycerol, polyethylene glycol 400, tetrahydrofurfuryl alcohol and diethylene glycol monoethyl ether. Preferred solubilizers include, e.g., glycerol-, polyglycerol-, propylene glycol- and polyoxyethylene-esters of medium chain and/or long chain fatty acids, such as glyceryl monolinoleate, medium chain glycerides and medium chain triglycerides, non-ionic solubilizers made by reacting castor oil with ethylene oxide, and any mixtures thereof which may further contain fatty acids or fatty alcohols; cellulose and methylcellulose and derivatives thereof such as hydroxypropylcellulose and hypromellose acetate succinate; various cyclodextrins and derivatives thereof; non-ionic tri-block copolymers having a central hydrophobic chain of polyoxypropylene flanked by two hydrophilic chains of polyoxyethylene known as poloxamers; water-soluble derivatives of vitamin E; pharmaceutical graded or agglomerated spherical isomalt; a polyethylene glycol, polyvinyl acetate and polyvinylcaprolactame-based graft copolymer, also abbreviated as PVAc-PVCap-PEG and known as Soluplus®; vinylpyrrolidone-vinyl acetate copolymers such as Kollidon® VA64; purified grades of naturally derived castor oil, of polyethylene glycol 400, of polyoxyethylene sorbitan monooleate (such as polysorbate 80) or of propylene glycols; diethylene glycol monoethyl ether; glucono-delta-lactone; maize and potato starch; as well as any of the below mentioned soluble polyvinylpyrrolidones, but also insoluble/cross-linked polyvinylpyrrolidones such as crospovidones. Also the permeation enhancers mentioned above can act as solubilizers. Furthermore, also the film-forming agents described below may act at the same time as solubilizers and vice versa.

In certain embodiments, the huperzine A-containing layer comprises a film-forming agent. It is to be understood that the above mentioned solubilizers may also act as film-forming agents and control cohesion. Suitable examples of film-forming agents include polyvinylpyrrolidone and in particular soluble polyvinylpyrrolidone, Soluplus® (Soluplus® is a polyethylene glycol, polyvinyl acetate and polyvinylcaprolactame-based graft copolymer, also abbreviated as PVAc-PVCap-PEG), polyethylene glycol, cellulose derivatives and other hydrophilic additives. Soluplus may be selected from polyethylene glycol, polyvinyl acetate and polyvinylcaprolactame-based graft copolymers (PVAc-PVCap-PEG).

In case the huperzine A-containing matrix layer is required to have self-adhesive properties and one or more polymers is/are selected which does/do not provide sufficient self-adhesive properties, a tackifier may be added. The tackifier may be selected from polyvinyl alcohol, alginate, guar gum, triglycerides, dipropylene glycol, resins, resin esters, terpenes and derivatives thereof, ethylene vinyl acetate adhesives, dimethylpolysiloxanes and polybutenes.

In one embodiment, the huperzine A-containing matrix layer further comprises a softener. Exemplary softeners include linear or branched, saturated or unsaturated alcohols having 6 to 20 carbon atoms.

The huperzine A-containing matrix layer according to the invention may comprise a plasticizer. Preferably, the plasticizer is selected from glycerol, glycerol esters, glycol derivatives, fatty acid ester, acid ester and sugar alcohols.

In one embodiment, the huperzine A-containing matrix layer layer further comprises a stabilizing agent. Stabilizing agents include tocopherol and ester derivatives thereof and ascorbic acid and ester derivatives thereof. Further stabilizing agents include sodium metabisulfite, ascorbyl esters of fatty acids such as ascorbyl palmitate, ascorbic acid, butylated hydroxytoluene, tocopherol, tocopheryl acetate and tocopheryl linoleate.

In one embodiment, the huperzine A-containing matrix layer further comprises a pH regulator. Suitable pH regulators include mild acids and bases including amine derivatives, inorganic alkali derivatives, and polymers with basic or acidic functionality.

In one embodiment, the huperzine A-containing matrix layer further comprises a preservative. Suitable preservatives include parabens, formaldehyde releasers, isothiazolinones, and phenoxyethanol.

In one embodiment, the huperzine A-containing matrix layer further comprises a substance for skincare. Such substances may be used to avoid or reduce skin irritation as detectable by the dermal response score. Suitable substances for skincare include lecithinm aloe, phosphatides, sterol compounds such as cholesterol, dexpanthenol, alpha-bisabolol, and antihistamines.

In one embodiment, the huperzine A-containing matrix layer further comprises a filler. Fillers such as silica gels, titanium dioxide and zinc oxide may be used in conjunction with the polymer in order to influence certain physical parameters, such as cohesion and bond strength, in the desired way.

### Release Characteristics

It has been found that the TTS according to the present invention is suitable for the transdermal administration of huperzine A to the systemic circulation, while at the same time providing good adhesive properties.

The TTS according to the present invention is suitable for the transdermal administration of huperzine A to the systemic circulation for a predefined extended period of time, preferably for at least 24 hours, at least 84 hours or 168 hours.

Preferably, the TTS provides therapeutically effective plasma concentrations of huperzine A within less than 10 hours, preferably less than 8 hours after application of the TTS to the skin.

Preferably, the TTS provides, after a steady state of the plasma concentration is reached, a therapeutically effective steady state plasma concentration of huperzine A for at least 12 hours, preferably at least 18 hours, more preferably at least 20 hours, provided that the TTS is administered to the skin for a sufficient time, e.g. for at least 24 hours, so that the steady state can be reached and maintained.

In one embodiment, the TTS provides a cumulative permeated amount of huperzine A as measured in a Franz diffusion cell with human skin of about 10 to 290 μg/cm² over a time period of about 24 hours.

In another embodiment, the TTS provides a cumulative permeated amount of huperzine A as measured in a Franz diffusion cell with human skin of about 40-510 μg/cm² over a time period of about 72 hours.

### Method of Treatment/Medical Use

In accordance with a specific aspect of the present invention, the TTS according to the invention is for use in a method of treatment. In particular, the TTS according to the invention is for use in a method of treating a human patient.

The invention is in particular related to a method of treatment, including applying a transdermal therapeutic system according to the invention to the skin of a patient.

In certain embodiments, the TTS according to the invention is for use in a method of treating Alzheimer's disease or any disease related to cognitive dysfunction, preferably including applying a transdermal therapeutic system according to the invention to the skin of a patient.

The method of treatment as outlined above may in particular include applying a transdermal therapeutic system according to the invention for at least 24 hours or 1 day, at least 48 hours or 2 days, at least 72 hours or 3 days, at least 84 hours or 3.5 days or at least 168 hours or 7 days to the skin of a patient.

The method of treatment as outlined above may also include applying a transdermal therapeutic system according to the invention for up to 168 hours or 7 days, up to 120 hours or 5 days, up to 96 hours or 4 days, or up to 24 hours or 1 day to the skin of a patient.

Preferably, the TTS according to the invention is applied to the skin of the patient for at least 24 hours (1 day), or 84 hours (3.5 days), or 168 hours (7 days) Specifically, the method of treatment as outlined above may in particular include applying a transdermal therapeutic system according to the invention for about 24 hours or 1 day, about 84 hours or 3.5 days, or about 168 hours or 7 days to the skin of a patient.

Accordingly, the invention is also related to a method of treatment in an around-the-clock treatment with a once-a-day TTS exchange mode (dosing interval of 24 hours or 1 day), a twice-a-week TTS exchange mode (dosing interval of 84 hours or 3.5 days) or a once-a-week TTS exchange mode (dosing interval of 168 hours or 7 days).

Accordingly the invention is also related to TTS for use in a method of treatment, and in particular for use in a method of treating Alzheimer's disease or any disease related to cognitive dysfunction in an around-the-clock treatment with a once-a-day TTS exchange mode (dosing interval of 24 hours or 1 day), a twice-a-week TTS exchange mode (dosing interval of 84 hours or 3.5 days) or a once-a-week TTS exchange mode (dosing interval of 168 hours or 7 days).

### Process of Manufacture

The invention further relates to a process of manufacture of an huperzine A-containing layer for use in a transdermal therapeutic system and a corresponding huperzine A-containing layer structure and a corresponding TTS.

The process of manufacture of a hhuperzine A-containing layer for use in a transdermal therapeutic system according to the invention comprises the steps of:
1) combining at least the components huperzine A, the at least one polymer and the at least one additive in a solvent to obtain a coating composition;
2) coating the coating composition onto the backing layer or release liner; and
3) drying the coated coating composition to form the huperzine A-containing layer.

In this process of manufacture, the huperzine A and at least one additive is preferably combined in the solvent first and subsequently the at least one polymer is added.

It is to be understood that in step 1), also further ingredients as defined above may be added.

The solvent in step 1) of the process is either added and/or present because the one or more polymer(s) are provided in the form of a solution. The solvent is preferably selected from alcoholic solvents, in particular methanol, ethanol, isopropanol and mixtures thereof, or from non-alcoholic solvents, in particular ethyl acetate, hexane, heptane, petroleum ether, toluene, and mixtures thereof. Preferably, the solvent is selected from non-alcoholic solvents and is most preferably ethyl acetate or n-heptane. In a particularly preferred embodiment, the solvent is ethyl acetate.

In one embodiment, the at least on polymer as defined above, is provided as a solution, wherein the solvent is ethyl acetate or n-heptane, and is preferably ethyl acetate. In another embodiment, the polymer is provided in the form of particles.

If the polymer is provided as a solution, the solids content is preferably from 25% to 60% weight, more preferably from 30% to 50% by weight, and even more preferably from 35% to 40% by weight.

In step 3), drying is performed preferably at a temperature of from 15 to 90° C., more preferably from 30 to 80° C., even more preferably from 50 to 70° C. In a particularly preferred embodiment, drying is first performed at room temperature followed by drying at a temperature of from 50 to 70° C.

### EXAMPLES

The present invention will now be more fully described with reference to the accompanying examples. It should be understood, however, that the following description is illustrative only and should not be taken in any way as a restriction of the invention. Numerical values provided in the examples regarding the amount of ingredients in the composition or the area weight may vary slightly due to manufacturing variability. If not specifically otherwise stated, all ingredients of the TTS as listed in the tables below are commercially available.

### Example 1A-1F

### Coating Composition

The formulations of the huperzine A-containing coating compositions of Examples 1A-1F are summarized in Tables 1.1-1.3 below. The solids %-values refer to the solid amounts (Amt) in % by weight.

TABLE 1.1

| | Ex. 1A | | Ex. 1B | |
|---|---|---|---|---|
| | Amt. | Solids | Amt. | Solids |
| Ingredient (Trade Name) | [g] | [%] | [g] | [%] |
| Huperzine A | 0.38 | 5.01 | 0.38 | 4.99 |
| Polyacrylate adhesive in ethyl | 6.36 | 84.98 | — | — |
| acetate Solids content of 39% | | | | |
| by weight (DURO-TAK ™ 87- | | | | |
| 4287 from Henkel) | | | | |
| Polyacrylate adhesive in ethyl | — | — | 6.38 | 84.87 |
| acetate Solids content of 36.5% | | | | |
| by weight (DURO-TAK ™ 87- | | | | |
| 9301 from Henkel) | | | | |
| Polyvinylpyrrolidone K90F | 0.75 | 10.01 | 0.76 | 10.14 |
| Total | 7.49 | 100.0 | 7.52 | 100.0 |

| | | |
|---|---|---|
| Area Weight [g/m²] | 102.85 | 101.95 |
| Loading API [μg/cm²] | 515.3 | 508.7 |

TABLE 1.2

| | Ex. 1C | | Ex. 1D | |
|---|---|---|---|---|
| | Amt. | Solids | Amt. | Solids |
| Ingredient (Trade Name) | [g] | [%] | [g] | [%] |
| Huperzine A | 0.37 | 5.00 | 0.37 | 4.99 |
| Polyacrylate adhesive in ethyl | — | — | 6.00 | 80.01 |
| acetate Solids content of 39% | | | | |
| by weight (DURO-TAK ™ 87- | | | | |
| 4287 from Henkel) | | | | |
| Loctite DURO-TAK AH 115 | 6.37 | 84.97 | — | — |
| Polyvinylpyrrolidone K90F | 0.75 | 10.04 | 0.74 | 9.91 |
| Lauryl lactate | — | — | 0.38 | 5.09 |
| Total | 7.49 | 100.0 | 7.49 | 100.0 |

| | | |
|---|---|---|
| Area Weight [g/m²] | 98.98 | 101.26 |
| Loading API [μg/cm²] | 494.9 | 505.3 |

TABLE 1.3

| | Ex. 1E | | Ex. 1F | |
|---|---|---|---|---|
| | Amt. | Solids | Amt. | Solids |
| Ingredient (Trade Name) | [g] | [%] | [g] | [%] |
| Huperzine A | 0.38 | 5.01 | 0.38 | 5.01 |
| Polyacrylate adhesive in ethyl | 6.01 | 79.94 | 6.00 | 79.90 |
| acetate Solids content of 39% | | | | |
| by weight (DURO-TAK ™ 87- | | | | |
| 4287 from Henkel) | | | | |
| Polyvinylpyrrolidone K90F | 0.75 | 9.94 | 0.75 | 10.04 |
| Transcutol | 0.38 | 5.11 | — | — |
| Methyl laurate | — | — | 0.38 | 5.05 |
| Total | 7.52 | 100.0 | 7.51 | 100.0 |

| | | |
|---|---|---|
| Area Weight [g/m²] | 99.29 | 101.40 |
| Loading API [μg/cm²] | 497.4 | 508.0 |

### Preparation of the Coating Composition

Drug substance, used enhancers and crystallization inhibitors were dispersed in the solvent ethyl acetate. Then the adhesives were added. The resulting mass was stirred for appr. 2 hours at 200 rpm.

### Coating of the Coating Composition

The resulting huperzine A-containing coating composition was coated on a polyethylene terephthalate film which may function as a release liner using for example a film applicator from the company Erichsen according to the solid content of the mixture under consideration of the desired coating dry weight and dried for appr. 15 min at room temperature and appr. 15 min at 60° C. Depending on the target area weight the corresponding film applicator gap is 380 μm.

The coating thickness was chosen such that removal of the solvents results in an area weight of the huperzine A-containing layer of approx. 102.85 (Ex. 1A) and 101.95 (Ex. 1B), 98.98 (Ex. 1C), 101.26 (Ex. 1D), 99.29 (Ex. 1E) and 101.40 (Ex. 1F) g/m². The dried film was then laminated to a backing layer (PET 23 μm) to provide a huperzine A-containing self-adhesive layer structure.

### Preparation of the TTS (Concerning all Examples)

The individual systems (TTS) were then punched out from the huperzine A-containing self-adhesive layer structure obtained as described above. Then, the TTS were sealed into pouches of the primary packaging material.

### Measurement of Skin Permeation

The permeated amount of TTS prepared according to Examples 1A-F was determined in accordance with the OECD Guideline (adopted Apr. 13, 2004) carried out with a 7.0 mL Franz diffusion cell. Human skin (female) was used. A dermatome was used to prepare skin to a thickness of 500 μm, with an intact epidermis for all TTS. Diecuts with an area of release of 1.157 cm² were punched from the TTS. The huperzine A permeated amount in the receptor medium of the Franz diffusion cell (phosphate buffer pH 5.5 with 0.1% sodium azide as antibacteriological agent) at a temperature of 32±1° C. was measured and the corresponding cumulative permeated amount was calculated.

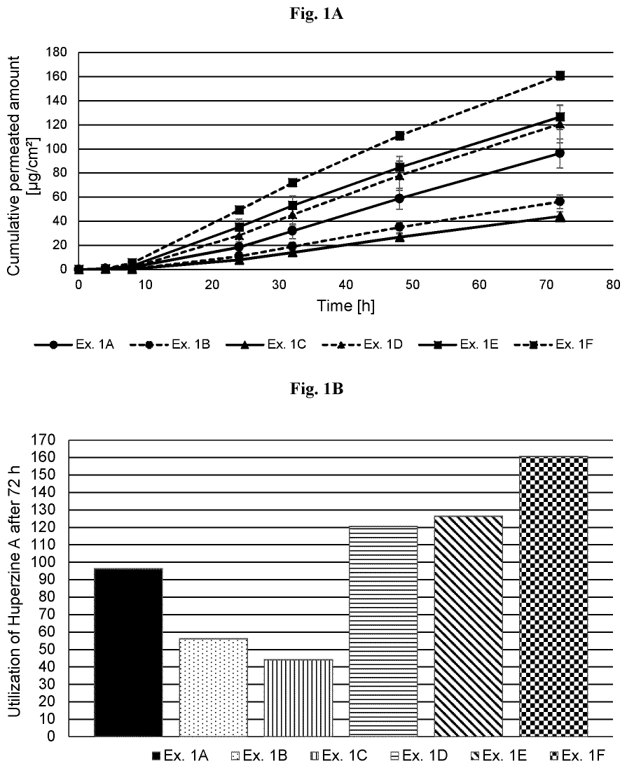

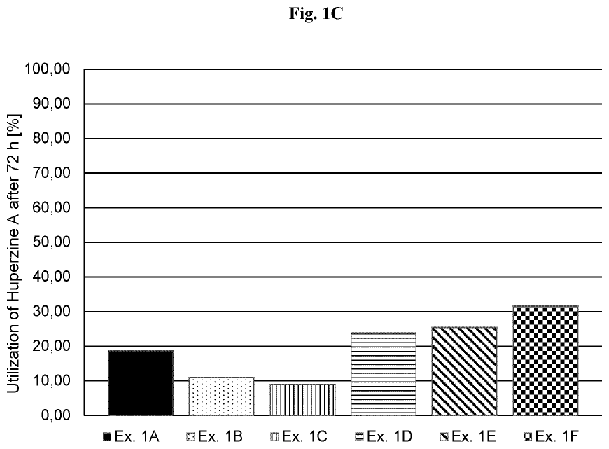

The results are shown in Table 1.4 and 1.5 and FIG. 1A-C.

TABLE 1.4
Cumulative permeated amount with SD [μg/cm²]

| Elapsed | Ex. 1A (n = 3) | | Ex. 1B (n = 3) | | Ex. 1C (n = 3) | |
|---|---|---|---|---|---|---|
| time [h] | Amount | SD | Amount | SD | Amount | SD |
| 4 | 0.7 | 0.92 | 0.08 | 0.06 | 0.09 | 0.01 |
| 8 | 1.69 | 1.88 | 0.33 | 0.07 | 0.34 | 0.07 |
| 24 | 18.38 | 4.64 | 10.87 | 2.31 | 7.87 | 1.55 |
| 32 | 31.74 | 6.09 | 18.98 | 3.27 | 14.03 | 2.22 |
| 48 | 58.62 | 8.79 | 34.89 | 4.47 | 26.73 | 2.98 |
| 72 | 96.22 | 12.11 | 56.13 | 5.69 | 44.09 | 3.73 |

TABLE 1.5
Cumulative permeated amount with SD [μg/cm²]

| Elapsed | Ex. 1D (n = 3) | | Ex. 1E (n = 3) | | Ex. 1F (n = 3) | |
|---|---|---|---|---|---|---|
| time [h] | Amount | SD | Amount | SD | Amount | SD |
| 4 | 0.2 | 0.06 | 0.37 | 0.25 | 0.6 | 0.04 |
| 8 | 1.64 | 0.76 | 2.93 | 1.4 | 5.31 | 0.39 |
| 24 | 28.17 | 7.01 | 35.06 | 6.66 | 49.14 | 2.24 |
| 32 | 45.44 | 9.04 | 52.9 | 8.02 | 71.83 | 2.86 |
| 48 | 77.72 | 12.13 | 84.51 | 9.35 | 110.8 | 3.44 |
| 72 | 120.53 | 15.46 | 126.31 | 10.13 | 160.67 | 3.64 |

The utilization of huperzine A at 72 h was calculated based on the cumulative permeated amount at 72 h and the initial huperzine A content. The results are shown in Table 1.6 and in FIG. 1A-C.

TABLE 1.6
Utilization of Huperzine A after 72 h [%]

| Ex. 1A | Ex. 1B | Ex. 1C | Ex. 1D | Ex. 1E | Ex. 1F |
|---|---|---|---|---|---|
| 18.7 | 11.0 | 8.9 | 23.8 | 25.4 | 31.6 |

### Example 2A-2E

### Coating Composition

The formulations of the huperzine A-containing coating compositions of Examples 2A-2E are summarized in Tables 2.1-2.3 below. The solids %-values refer to the solid amounts (Amt) in % by weight.

TABLE 2.1

| | Ex. 2A | | Ex. 2B | |
|---|---|---|---|---|
| | Amt. | Solids | Amt. | Solids |
| Ingredient (Trade Name) | [g] | [%] | [g] | [%] |
| Huperzine A | 0.57 | 7.60 | 0.38 | 5.03 |
| Polyacrylate adhesive in ethyl | 5.81 | 77.03 | 5.63 | 74.80 |
| acetate Solids content of 39% | | | | |
| by weight (DURO-TAK ™ 87- | | | | |
| 4287 from Henkel) | | | | |
| Polyvinylpyrrolidone K90F | 0.76 | 10.10 | 0.75 | 9.99 |
| Methyl laurate | 0.40 | 5.27 | 0.38 | 5.05 |
| 1-Dodecanol | — | — | 0.39 | 5.13 |
| Total | 7.54 | 100.0 | 7.53 | 100.0 |

| | | |
|---|---|---|
| Area Weight [g/m²] | 101.03 | 100.61 |
| Loading API [μg/cm²] | 767.8 | 506.1 |

TABLE 2.2

| | Ex. 2C | | Ex. 2D | |
|---|---|---|---|---|
| | Amt. | Solids | Amt. | Solids |
| Ingredient (Trade Name) | [g] | [%] | [g] | [%] |
| Huperzine A | 0.76 | 9.84 | 0.57 | 7.55 |
| Polyacrylate adhesive in ethyl | 5.82 | 75.49 | 5.82 | 77.44 |
| acetate Solids content of 39% | | | | |
| by weight (DURO-TAK ™ 87- | | | | |
| 4287 from Henkel) | | | | |
| Polyvinylpyrrolidone K90F | 0.75 | 9.72 | 0.75 | 10.04 |
| Methyl laurate | 0.38 | 4.96 | 0.37 | 4.97 |
| Total | 7.71 | 100.0 | 7.51 | 100.0 |

| | | |
|---|---|---|
| Area Weight [g/m²] | 103.68 | 140.80 |
| Loading API [μg/cm²] | 1020.2 | 1063.0 |

TABLE 2.3

Ex. 2E

| | Amt. | Solids |
|---|---|---|
| Ingredient (Trade Name) | [g] | [%] |
| Huperzine A | 0.38 | 5.04 |
| Polyacrylate adhesive in ethyl acetate | 5.61 | 74.90 |
| Solids content of 39% by weight | | |
| (DURO-TAK ™ 87-4287 from Henkel) | | |
| Polyvinylpyrrolidone K90F | 0.75 | 10.04 |
| 1-Dodecanol | 0.38 | 5.01 |
| Methyl laurate | 0.37 | 5.01 |
| Total | 7.49 | 100.0 |

| | |
|---|---|
| Area Weight [g/m²] | 73.78 |
| Loading API [μg/cm²] | 371.9 |

### Preparation of the Coating Composition

Drug substance, used enhancers and crystallization inhibitor were dispersed in the solvent ethyl acetate. Then the adhesive was added. The resulting mass was stirred for approximately 2 hours at 200 upm.

### Coating of the Coating Composition

10 The resulting huperzine A-containing coating composition was coated on a polyethylene terephthalate film, which may function as a release liner using for example a film applicator from the company Erichsen according to the solid content of the mixture under consideration of the desired coating dry weight and dried for appr. 15 min at room temperature and appr. 15 min at 60° C. Depending on the target area weight the corresponding film applicator gap is varying between 245 and 590 μm.

The coating thickness was chosen such that removal of the solvents results in an area weight of the huperzine A-containing layer of approx. 101.03 (Ex. 2A), 100.61 (Ex. 2B), 103.68 (Ex. 2C), 140.80 (Ex. 2D) and 73.78 (Ex. 2E) g/m². The dried film was then laminated to a backing layer (PET 23 μm) to provide a huperzine A-containing self-adhesive layer structure.

### Preparation of the TTS (Concerning all Examples)

The individual systems (TTS) were then punched out from the huperzine A-containing self-adhesive layer structure obtained as described above. Then, the TTS were sealed into pouches of the primary packaging material.

### Measurement of Skin Permeation

The permeated amount of TTS prepared according to Examples 2A-E was determined in accordance with the OECD Guideline (adopted Apr. 13, 2004) carried out with a 7.0 mL Franz diffusion cell. Human skin (female) was used. A dermatome was used to prepare skin to a thickness of 500 μm, with an intact epidermis for all TTS. Diecuts with an area of release of 1.152 cm² were punched from the TTS. The huperzine A permeated amount in the receptor medium of the Franz diffusion cell (phosphate buffer pH 5.5 with 0.1% sodium azide as antibacteriological agent) at a temperature of 32±1° C. was measured and the corresponding cumulative permeated amount was calculated.

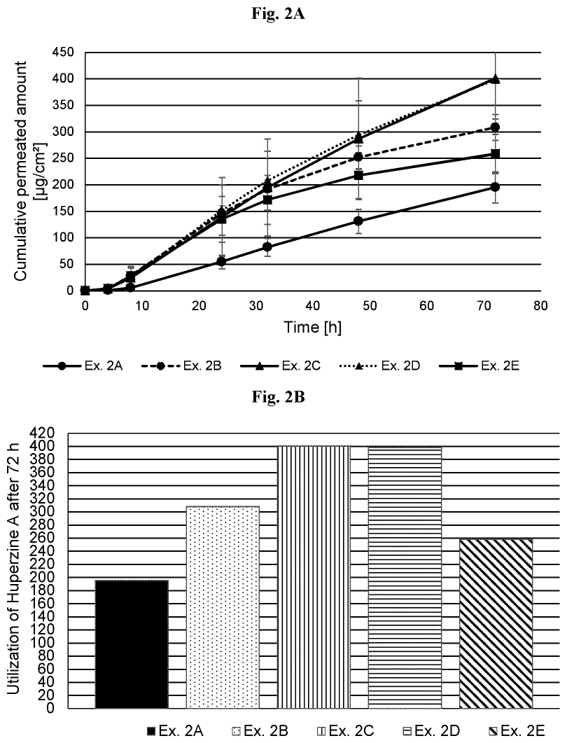

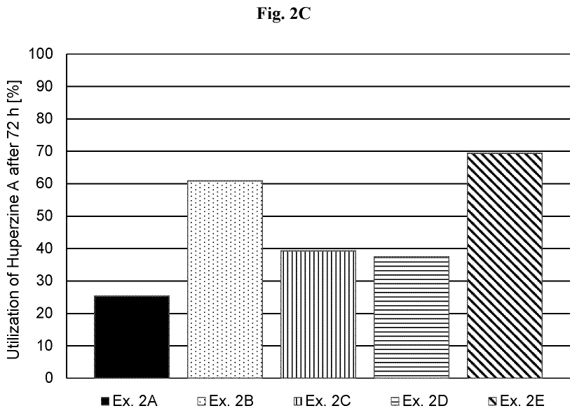

The results are shown in Table 2.4 and 2.5 and FIG. 2A-C.

TABLE 2.4
Cumulative permeated amount with SD [μg/cm²]

| Elapsed | Ex. 2A (n = 3) | | Ex. 2B (n = 3) | | Ex. 2C (n = 3) | |
|---|---|---|---|---|---|---|
| time [h] | Amount | SD | Amount | SD | Amount | SD |
| 4 | 0.58 | 0.35 | 3.8 | 2.01 | 3.96 | 4.49 |
| 8 | 5.11 | 2.05 | 28.06 | 5.74 | 24.36 | 21.4 |
| 24 | 54.32 | 12.7 | 146.32 | 13.76 | 139.46 | 74.28 |
| 32 | 82.01 | 16.89 | 191.66 | 17.1 | 194.97 | 91.77 |
| 48 | 130.84 | 22.83 | 251.86 | 21.43 | 286.71 | 114.63 |
| 72 | 195.19 | 29.51 | 308.28 | 24.4 | 400.71 | 141.01 |

TABLE 2.5
Cumulative permeated amount with SD [μg/cm²]

| | | |
|---|---|---|
| Elapsed | Ex. 2D (n = 3) | Ex. 2E (n = 3) |

| time [h] | Amount | SD | Amount | SD |
|---|---|---|---|---|
| 4 | 3.00 | 2.77 | 3.97 | 3.07 |
| 8 | 25.74 | 16.73 | 28.03 | 16.4 |
| 24 | 152 | 47.39 | 134.96 | 43.22 |
| 32 | 207.73 | 55.53 | 171.47 | 46.05 |
| 48 | 293.59 | 64.77 | 217.53 | 43.55 |
| 72 | 398.34 | 73.89 | 258.2 | 36.7 |

The utilization of huperzine A at 72 h was calculated based on the cumulative permeated amount at 72 h and the initial huperzine A content. The results are shown in Table 2.6 and in FIG. 2A-C.

TABLE 2.6
Utilization of Huperzine A after 72 h [%]

| Ex. 2A | Ex. 2B | Ex. 2C | Ex. 2D | Ex. 2E |
|---|---|---|---|---|
| 25.4 | 60.9 | 39.3 | 37.5 | 69.4 |

### Example 3A-3E

### Coating Composition

The formulations of the huperzine A-containing coating compositions of Examples 3A-3E are summarized in Tables 3.1-3.3 below. The solids %-values refer to the solid amounts (Amt) in % by weight.

TABLE 3.1

| | Ex. 3A | | Ex. 3B | |
|---|---|---|---|---|
| | Amt. | Solids | Amt. | Solids |
| Ingredient (Trade Name) | [g] | [%] | [g] | [%] |
| Huperzine A | 0.38 | 5.08 | 0.38 | 5.06 |
| Polyacrylate adhesive in ethyl | 5.98 | 79.87 | 6.38 | 84.73 |
| acetate Solids content of 39% | | | | |
| by weight (DURO-TAK ™ 87- | | | | |
| 4287 from Henkel) | | | | |
| Polyvinylpyrrolidone K90F | 0.75 | 9.95 | 0.38 | 5.03 |
| Methyl laurate | 0.38 | 5.10 | 0.39 | 5.19 |
| Total | 7.49 | 100.0 | 7.53 | 100.0 |

| | | |
|---|---|---|
| Area Weight [g/m²] | 102.77 | 99.78 |
| Loading API [μg/cm²] | 522.1 | 504.9 |

TABLE 3.2

| | Ex. 3C | | Ex. 3D | |
|---|---|---|---|---|
| | Amt. | Solids | Amt. | Solids |
| Ingredient (Trade Name) | [g] | [%] | [g] | [%] |
| Huperzine A | 0.57 | 7.60 | 0.38 | 5.05 |
| Polyacrylate adhesive in ethyl | 5.81 | 77.03 | 5.62 | 74.72 |
| acetate Solids content of 39% | | | | |
| by weight (DURO-TAK ™ 87- | | | | |
| 4287 from Henkel) | | | | |
| Polyvinylpyrrolidone K90F | 0.76 | 10.10 | 0.75 | 10.02 |
| Methyl laurate | 0.40 | 5.27 | 0.38 | 5.06 |
| Capryol 90 | — | — | 0.39 | 5.15 |
| Total | 7.54 | 100.0 | 7.52 | 100.0 |

| | | |
|---|---|---|
| Area Weight [g/m²] | 101.03 | 100.48 |
| Loading API [μg/cm²] | 767.8 | 507.4 |

TABLE 3.3

Ex. 3E

| | Amt. | Solids |
|---|---|---|
| Ingredient (Trade Name) | [g] | [%] |
| Huperzine A | 0.38 | 5.03 |
| Polyacrylate adhesive in ethyl acetate | 5.63 | 74.80 |
| Solids content of 39% by weight | | |
| (DURO-TAK ™ 87-4287 from Henkel) | | |
| Polyvinylpyrrolidone K90F | 0.75 | 9.99 |
| 1-Dodecanol | 0.39 | 5.13 |
| Methyl laurate | 0.38 | 5.05 |
| Total | 7.53 | 100.0 |

| | |
|---|---|
| Area Weight [g/m²] | 100.61 |
| Loading API [μg/cm²] | 506.1 |

### Preparation of the Coating Composition

Drug substance, used enhancers and crystallization inhibitor were dispersed in the solvent ethyl acetate. Then the adhesive was added. The resulting mass was stirred for appr. 2 hours at 200 upm.

### Coating of the Coating Composition

The resulting huperzine A-containing coating composition was coated on a polyethylene terephthalate film which may function as a release liner using for example a film applicator from the company Erichsen according to the solid content of the mixture under consideration of the desired coating dry weight and dried for appr. 15 min at room temperature and appr. 15 min at 60° C. Depending on the target area weight the corresponding film applicator gap is 380 μm.

The coating thickness was chosen such that removal of the solvents results in an area weight of the huperzine A-containing layer of approx. 102.77 (Ex. 3A) and 99.78 (Ex. 3B), 101.03 (Ex. 3C), 100.48 (Ex. 3D) and 100.61 (Ex. 3E) g/m². The dried film was then laminated to a backing layer (PET 23 μm) to provide a huperzine A-containing self-adhesive layer structure.

### Preparation of the TTS (Concerning all Examples)

The individual systems (TTS) were then punched out from the huperzine A-containing self-adhesive layer structure obtained as described above. Then, the TTS were sealed into pouches of the primary packaging material.

### Measurement of Skin Permeation

The permeated amount of TTS prepared according to Examples 3A-E was determined in accordance with the OECD Guideline (adopted Apr. 13, 2004) carried out with a 7.0 mL Franz diffusion cell. Human skin (female) was used. A dermatome was used to prepare skin to a thickness of 500 μm, with an intact epidermis for all TTS. Diecuts with an area of release of 1.152 cm² were punched from the TTS. The huperzine A permeated amount in the receptor medium of the Franz diffusion cell (phosphate buffer pH 5.5 with 0.1% sodium azide as antibacteriological agent) at a temperature of 32±1° C. was measured and the corresponding cumulative permeated amount was calculated.

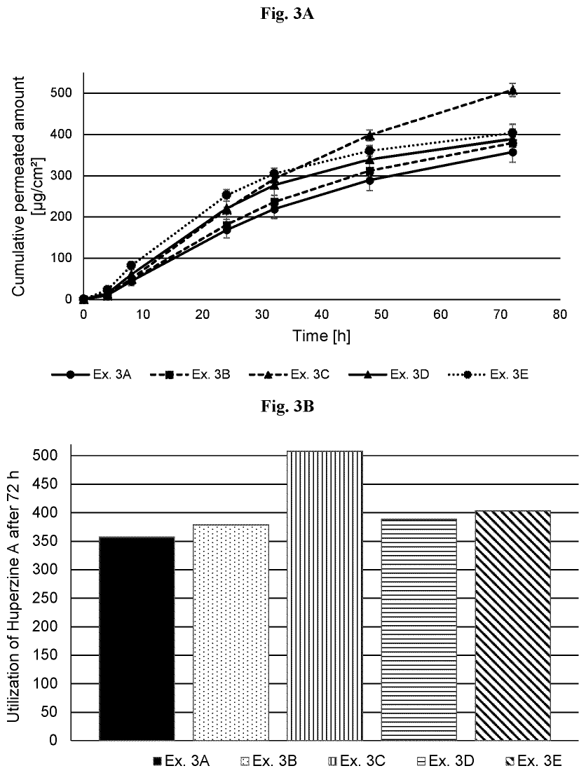

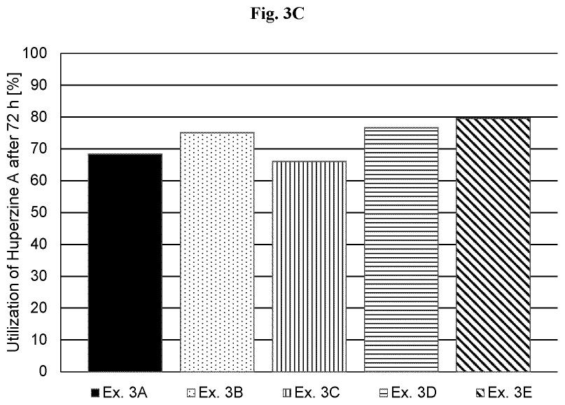

The results are shown in Table 3.4 and 3.5 and FIG. 3A-C.

TABLE 3.4
Cumulative permeated amount with SD [μg/cm²]

| Elapsed | Ex. 3A (n = 3) | | Ex. 3B (n = 3) | | Ex. 3C (n = 3) | |
|---|---|---|---|---|---|---|
| time [h] | Amount | SD | Amount | SD | Amount | SD |
| 4 | 10.24 | 3.57 | 10.71 | 2.18 | 9.71 | 1.22 |
| 8 | 43.96 | 10.04 | 46.78 | 6.3 | 49.8 | 0.94 |
| 24 | 168.21 | 19.16 | 180.53 | 13.45 | 217.95 | 5.8 |
| 32 | 218.76 | 22.34 | 236.1 | 16.36 | 292.33 | 8.97 |
| 48 | 288.29 | 24.42 | 311.7 | 20.18 | 398.03 | 13.21 |
| 72 | 356.99 | 24.44 | 378.96 | 22.06 | 507.83 | 15.84 |

TABLE 3.5
Cumulative permeated amount with SD [μg/cm²]

| | | |
|---|---|---|
| Elapsed | Ex. 3D (n = 3) | Ex. 3E (n = 3) |

| time [h] | Amount | SD | Amount | SD |
|---|---|---|---|---|
| 4 | 14.06 | 6.15 | 23.11 | 5.56 |
| 8 | 60.36 | 14.75 | 81.4 | 9.9 |
| 24 | 220.86 | 26.72 | 252.45 | 14.12 |
| 32 | 276.83 | 31.26 | 304.58 | 14.05 |
| 48 | 338.94 | 34.92 | 359.84 | 12.36 |
| 72 | 388.81 | 36.08 | 403.06 | 10.03 |

The utilization of huperzine A at 72 h was calculated based on the cumulative permeated amount at 72 h and the initial huperzine A content. The results are shown in Table 3.6 and in FIG. 3A-C.

TABLE 3.6
Utilization of Huperzine A after 72 h [%]

| Ex. 3A | Ex. 3B | Ex. 3C | Ex. 3D | Ex. 3E |
|---|---|---|---|---|
| 68.4 | 75.1 | 66.1 | 76.6 | 79.6 |

### Example 4A-4F

### Coating Composition

The formulations of the huperzine A-containing coating compositions of Examples 4A-4F are summarized in Tables 4.1-4.3 below. The solids %-values refer to the solid amounts (Amt) in % by weight.

TABLE 4.1

| | Ex. 4A | | Ex. 4B | |
|---|---|---|---|---|
| | Amt. | Solids | Amt. | Solids |
| Ingredient (Trade Name) | [g] | [%] | [g] | [%] |
| Huperzine A | 0.21 | 2.06 | 0.20 | 2.02 |
| Polyisobutylene (DURO-TAK ™ | 7.81 | 77.92 | 1.50 | 82.95 |
| 87-6908 from Henkel) | | | | |
| Crospovidone | 2.00 | 20.02 | 1.50 | 15.03 |
| Total | 10.02 | 100.0 | 10.00 | 100.0 |

| | | |
|---|---|---|
| Area Weight [g/m²] | 97.60 | 95.70 |
| Loading API [μg/cm²] | 201.1 | 193.3 |

TABLE 4.2

| | Ex. 4C | | Ex. 4D | |
|---|---|---|---|---|
| | Amt. | Solids | Amt. | Solids |
| Ingredient (Trade Name) | [g] | [%] | [g] | [%] |
| Huperzine A | 0.20 | 2.05 | 0.30 | 2.02 |
| Polyisobutylene (DURO-TAK ™ | 8.81 | 87.91 | 11.69 | 77.88 |
| 87-6908 from Henkel) | | | | |
| Crospovidone | 1.01 | 10.04 | 3.02 | 20.10 |
| Total | 10.02 | 100.0 | 15.01 | 100.0 |

| | | |
|---|---|---|
| Area Weight [g/m²] | 95.90 | 139.60 |
| Loading API [μg/cm²] | 196.6 | 282.0 |

TABLE 4.3

| | Ex. 4E | | Ex. 4F | |
|---|---|---|---|---|
| | Amt. | Solids | Amt. | Solids |
| Ingredient (Trade Name) | [g] | [%] | [g] | [%] |
| Huperzine A | 0.20 | 2.04 | 0.20 | 2.03 |
| Polyisobutylene (DURO-TAK ™ | 7.31 | 72.90 | 8.31 | 82.87 |
| 87-6908 from Henkel) | | | | |
| Crospovidone | 2.00 | 19.97 | 1.00 | 9.98 |
| Levulinic acid | 0.51 | 5.09 | — | — |
| Methyl laurate | — | — | 0.51 | 5.11 |
| Total | 10.02 | 100.0 | 10.02 | 100.0 |

| | | |
|---|---|---|
| Area Weight [g/m²] | 99.70 | 92.90 |
| Loading API [μg/cm²] | 203.4 | 188.6 |

### Preparation of the Coating Composition

Drug substance, used enhancers and crystallization inhibitor were dispersed in the rsp. solvent mixture (ethyl acetate, isopropanol and n-heptane). Then the adhesive was added. The resulting mass was stirred for appr. 2 hours at 300 upm.

### Coating of the Coating Composition

The resulting huperzine A-containing coating composition was coated on a polyethylene terephthalate film which may function as a release liner using for example a film applicator from the company Erichsen according to the solid content of the mixture under consideration of the desired coating dry weight and dried for appr. 15 min at room temperature and appr. 15 min at 60° C. Depending on the target area weight the corresponding film applicator gap is 390 μm rsp. 570 μm for Ex. 4D

The coating thickness was chosen such that removal of the solution results in an area weight of the huperzine A-containing layer of approx. 97.60 (Ex. 4A), 95.70 (Ex. 4B), 95.90 (Ex. 4C), 139.60 (Ex. 4D), 99.70 (Ex. 4E) and 92.90 (Ex. 4F) g/m². The dried film was then laminated to a backing layer (PET 23 μm) to provide a huperzine A-containing self-adhesive layer structure.

### Preparation of the TTS (Concerning all Examples)

The individual systems (TTS) were then punched out from the huperzine A-containing self-adhesive layer structure obtained as described above. Then, the TTS were sealed into pouches of the primary packaging material.

### Measurement of Skin Permeation

The permeated amount of TTS prepared according to Examples 4A-F was determined in accordance with the OECD Guideline (adopted Apr. 13, 2004) carried out with a 7.0 mL Franz diffusion cell. Human skin (female) was used. A dermatome was used to prepare skin to a thickness of 500 μm, with an intact epidermis for all TTS. Diecuts with an area of release of 1.156 cm² were punched from the TTS. The huperzine A permeated amount in the receptor medium of the Franz diffusion cell (phosphate buffer pH 5.5 with 0.1% sodium azide as antibacteriological agent) at a temperature of 32±1° C. was measured and the corresponding cumulative permeated amount was calculated.

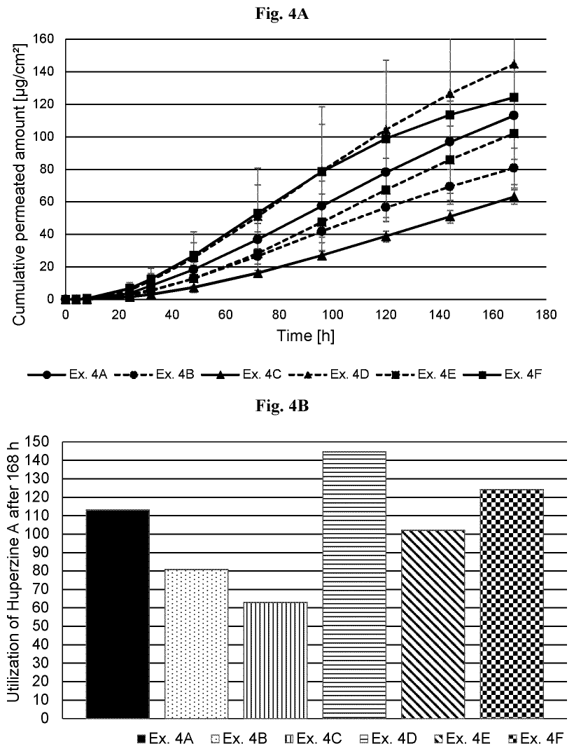

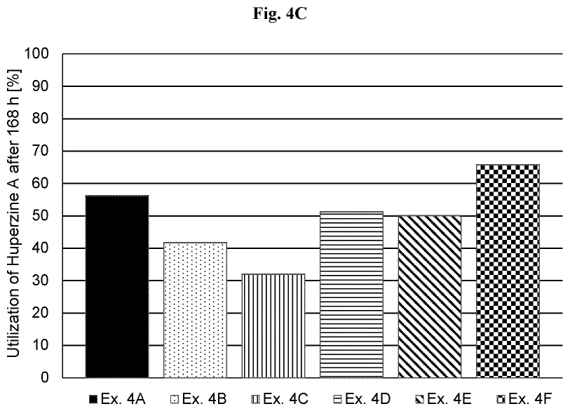

The results are shown in Table 4.4 and 4.5 and FIG. 4A-C.

TABLE 4.4
Cumulative permeated amount with SD [μg/cm²]

| Elapsed | Ex. 4A (n = 3) | | Ex. 4B (n = 3) | | Ex. 4C (n = 3) | |
|---|---|---|---|---|---|---|
| time [h] | Amount | SD | Amount | SD | Amount | SD |
| 4 | 0.00 | 0.00 | 0.00 | 0.00 | 0.00 | 0.00 |
| 8 | 0.09 | 0.16 | 0.07 | 0.06 | 0.04 | 0.06 |
| 24 | 3.82 | 1.08 | 2.33 | 0.42 | 1.54 | 0.29 |
| 32 | 8.4 | 2.39 | 5.25 | 0.97 | 3.05 | 0.45 |
| 48 | 18.28 | 4.6 | 12.69 | 2.02 | 7.31 | 0.89 |
| 72 | 36.85 | 9.93 | 26.5 | 4.7 | 16.14 | 1.74 |
| 96 | 57.34 | 15.54 | 41.77 | 6.88 | 26.96 | 2.52 |
| 120 | 78.02 | 21.49 | 56.5 | 8.4 | 38.72 | 3.28 |
| 144 | 96.7 | 25.19 | 69.26 | 10.64 | 50.89 | 3.84 |
| 168 | 112.96 | 26.78 | 80.72 | 12.24 | 63.01 | 4.33 |

TABLE 4.5
Cumulative permeated amount with SD [μg/cm²]

| Elapsed | Ex. 4D (n = 3) | | Ex. 4E (n = 3) | | Ex. 4F (n = 3) | |
|---|---|---|---|---|---|---|
| time [h] | Amount | SD | Amount | SD | Amount | SD |
| 4 | 0.00 | 0.00 | 0.00 | 0.00 | 0.1 | 0.09 |
| 8 | 0.14 | 0.16 | 0.23 | 0.26 | 0.58 | 0.35 |
| 24 | 5.57 | 2.4 | 3.01 | 2.98 | 6.77 | 3.7 |
| 32 | 11.45 | 4.43 | 5.58 | 4.78 | 12.51 | 6.82 |
| 48 | 25.76 | 9.18 | 12.76 | 8.39 | 27.05 | 14.52 |
| 72 | 50.87 | 19.57 | 28.24 | 13.36 | 52.96 | 27.81 |
| 96 | 78.82 | 28.88 | 47.5 | 17.28 | 78.41 | 40.00 |
| 120 | 104.34 | 35.74 | 67.36 | 19.5 | 98.76 | 48.35 |
| 144 | 126.48 | 39.91 | 85.89 | 20.68 | 113.38 | 52.36 |
| 168 | 144.64 | 40.47 | 102.01 | 20.39 | 124.12 | 53.39 |

The utilization of huperzine A at 168 h was calculated based on the cumulative permeated amount at 168 h and the initial huperzine A content. The results are shown in Table 4.6 and in FIG. 4A-C.

TABLE 4.6
Utilization of Huperzine A after 168 h [%]

| Ex. 4A | Ex. 4B | Ex. 4C | Ex. 4D | Ex. 4E | Ex. 4F |
|---|---|---|---|---|---|
| 56.2 | 41.7 | 32.0 | 51.3 | 50.1 | 65.8 |

### Example 5A-5F

### Coating Composition

The formulations of the huperzine A-containing coating compositions of Examples 5A-5F are summarized in Tables 5.1-5.3 below. The solids %-values refer to the solid amounts (Amt) in % by weight.

TABLE 5.1

| | Ex. 5A | | Ex. 5B | |
|---|---|---|---|---|
| | Amt. | Solids | Amt. | Solids |
| Ingredient (Trade Name) | [g] | [%] | [g] | [%] |
| Huperzine A | 0.21 | 2.07 | 0.20 | 2.03 |
| Polyisobutylene (DURO-TAK ™ | 7.78 | 77.94 | 7.30 | 72.84 |
| 87-6908 from Henkel) | | | | |
| Crospovidone | 2.00 | 19.99 | 2.01 | 20.01 |
| Methyl laurate | — | — | 0.51 | 5.12 |
| Total | 9.99 | 100.0 | 10.02 | 100.0 |

| | | |
|---|---|---|
| Area Weight [g/m²] | 104.72 | 103.35 |
| Loading API [μg/cm²] | 216.8 | 209.8 |

TABLE 5.2

| | Ex. 5C | | Ex. 5D | |
|---|---|---|---|---|
| | Amt. | Solids | Amt. | Solids |
| Ingredient (Trade Name) | [g] | [%] | [g] | [%] |
| Huperzine A | 0.20 | 2.04 | 0.20 | 2.02 |
| Polyisobutylene (DURO-TAK ™ | 7.30 | 72.63 | — | — |
| 87-6908 from Henkel) | | | | |
| Styrene Isoprene Styrene (SIS | — | — | 7.81 | 77.89 |
| 5229 P from JSR/Hydrogenated | | | | |
| Resin (Foral 85 from Pinova) 1 + 3 | | | | |
| Crospovidone | 2.04 | 20.33 | 2.01 | 20.09 |
| 1-Dodecanol | 0.50 | 5.00 | — | — |
| Total | 10.04 | 100.0 | 10.02 | 100.0 |

| | | |
|---|---|---|
| Area Weight [g/m²] | 99.39 | 103.52 |
| Loading API [μg/cm²] | 202.8 | 209.1 |

TABLE 5.3

| | Ex. 5E | | Ex. 5F | |
|---|---|---|---|---|
| | Amt. | Solids | Amt. | Solids |
| Ingredient (Trade Name) | [g] | [%] | [g] | [%] |
| Huperzine A | 0.21 | 2.05 | 0.20 | 2.01 |
| Styrene Isoprene Styrene (SIS | 7.30 | 72.96 | 7.30 | 72.85 |
| 5229 P from JSR/Hydrogenated | | | | |
| Resin (Foral 85 from Pinova) 1 + 3 | | | | |
| Crospovidone | 2.00 | 20.02 | 2.01 | 20.05 |
| 1-Dodecanol | — | — | 0.51 | 5.09 |
| Methyl laurate | 0.50 | 4.97 | — | — |
| Total | 10.01 | 100.0 | 10.02 | 100.0 |

| | | |
|---|---|---|
| Area Weight [g/m²] | 103.82 | 103.18 |
| Loading API [μg/cm²] | 212.8 | 207.4 |

### Preparation of the Coating Composition

Drug substance and used enhancers were dispersed in the solvent mixture ethylacetate/isopropanol for Ex. 5A-C and n-heptane for Ex.5D-F. Then the adhesives were added. The resulting mixture was stirred for appr. ½ hour at 400 upm Afterwards the crystallization inhibitor was added while stirring at 700 upm and the final mass was stirred for another 2 hours.

### Coating of the Coating Composition

The resulting huperzine A-containing coating composition was coated on a polyethylene terephthalate film which may function as a release liner using for example a film applicator from the company Erichsen according to the solid content of the mixture under consideration of the desired coating dry weight and dried for appr. 15 min at room temperature and appr. 15 min at 60° C. Depending on the target area weight the corresponding film applicator gap is 370 to 390 μm.

The coating thickness was chosen such that removal of the solution results in an area weight of the huperzine A-containing layer of approx. 104.72 (Ex. 5A), 103.35 (Ex. 5B), 99.39 (Ex. 5C), 103.52 (Ex. 5D), 103.82 (Ex. 5E) and 103.18 (Ex. 5F) g/m². The dried film was then laminated to a backing layer (PET 23 μm) to provide a huperzine A-containing self-adhesive layer structure.

### Preparation of the TTS (Concerning all Examples)

The individual systems (TTS) were then punched out from the huperzine A-containing self-adhesive layer structure obtained as described above. Then, the TTS were sealed into pouches of the primary packaging material.

### Measurement of Skin Permeation

The permeated amount of TTS prepared according to Examples 5A-F was determined in accordance with the OECD Guideline (adopted Apr. 13, 2004) carried out with a 7.0 mL Franz diffusion cell. Human skin (female) was used. A dermatome was used to prepare skin to a thickness of 500 μm, with an intact epidermis for all TTS. Diecuts with an area of release of 1.156 cm² were punched from the TTS. The huperzine A permeated amount in the receptor medium of the Franz diffusion cell (phosphate buffer pH 5.5 with 0.1% sodium azide as antibacteriological agent) at a temperature of 32±1° C. was measured and the corresponding cumulative permeated amount was calculated.

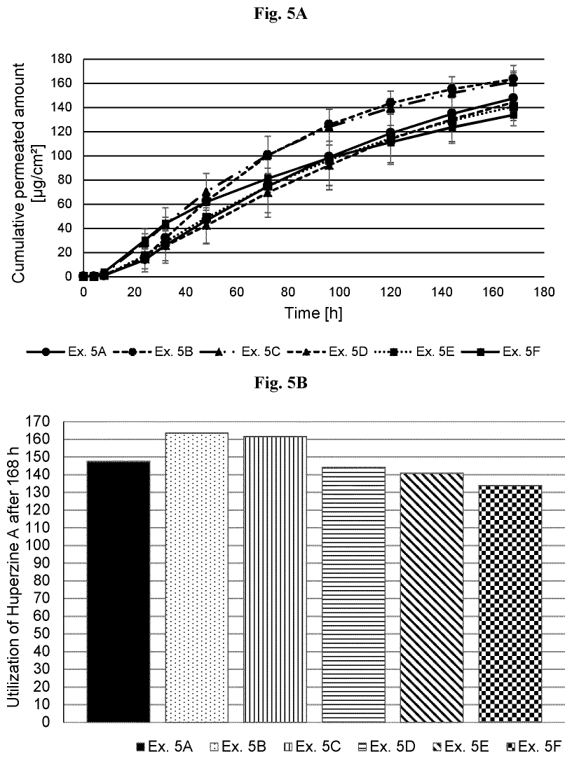

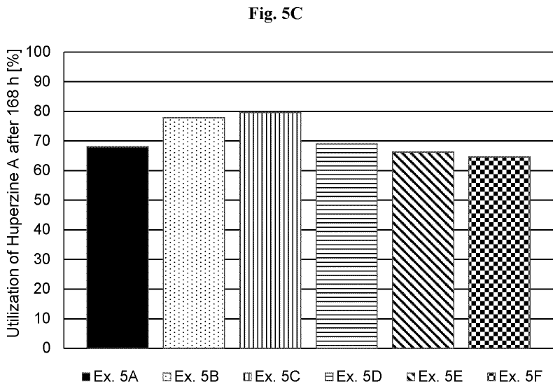

The results are shown in Table 5.4 and 5.5 and FIG. 5A-C.

TABLE 5.4
Cumulative permeated amount with SD [μg/cm²]

| Elapsed | Ex. 5A (n = 3) | | Ex. 5B (n = 3) | | Ex. 5C (n = 3) | |
|---|---|---|---|---|---|---|
| time [h] | Amount | SD | Amount | SD | Amount | SD |
| 4 | 0.1 | 0.09 | 0.00 | 0.00 | 0.15 | 0.16 |
| 8 | 0.77 | 0.79 | 0.62 | 0.07 | 2.31 | 1.8 |
| 24 | 14.01 | 9.88 | 16.23 | 0.68 | 28.36 | 11.4 |
| 32 | 25.96 | 14.76 | 31.84 | 0.93 | 43.6 | 13.55 |
| 48 | 46.51 | 19.23 | 62.33 | 1.92 | 70.24 | 15.27 |
| 72 | 74.84 | 21.92 | 100.37 | 2.11 | 100.74 | 15.49 |
| 96 | 98.97 | 23.45 | 125.7 | 2.5 | 123.78 | 14.83 |
| 120 | 118.62 | 23.99 | 143.42 | 4.19 | 139.48 | 14.34 |
| 144 | 134.67 | 24.09 | 155.1 | 4.75 | 152.06 | 13.48 |
| 168 | 147.54 | 22.69 | 163.51 | 5.17 | 161.51 | 13.38 |

TABLE 5.5
Cumulative permeated amount with SD [μg/cm²]

| Elapsed | Ex. 5D (n = 3) | | Ex. 5E (n = 3) | | Ex. 5F (n = 3) | |
|---|---|---|---|---|---|---|
| time [h] | Amount | SD | Amount | SD | Amount | SD |
| 4 | 0.24 | 0.26 | 0.00 | 0.00 | 0.27 | 0.24 |
| 8 | 1.45 | 1.52 | 0.91 | 0.01 | 3.56 | 1.94 |
| 24 | 14.74 | 8.04 | 17.35 | 1.81 | 30.11 | 5.55 |
| 32 | 25.52 | 12.35 | 28.72 | 2.56 | 43.94 | 5.26 |
| 48 | 42.41 | 14.58 | 48.7 | 3.82 | 61.7 | 4.52 |
| 72 | 69.49 | 20.27 | 74.82 | 4.85 | 81.22 | 4.21 |
| 96 | 92.13 | 20.08 | 96.48 | 4.92 | 98.18 | 2.33 |
| 120 | 113.84 | 20.7 | 114.51 | 4.29 | 111.04 | 2.44 |
| 144 | 130.08 | 18.1 | 128.98 | 3.23 | 123.44 | 1.05 |
| 168 | 144.24 | 14.87 | 140.79 | 1.87 | 133.87 | 1.04 |

The utilization of huperzine A at 168 h was calculated based on the cumulative permeated amount at 168 h and the initial huperzine A content. The results are shown in Table 5.6 and in FIG. 5A-C.

TABLE 5.6
Utilization of Huperzine A after 168 h [%]

| Ex. 5A | Ex. 5B | Ex. 5C | Ex. 5D | Ex. 5E | Ex. 5F |
|---|---|---|---|---|---|
| 68.0 | 77.9 | 79.6 | 69.0 | 66.2 | 64.6 |

### Comparative Examples 1A-B and Example 6A-6E

### Coating Composition

The formulations of the huperzine A-containing coating compositions of comparative Examples 1A-1B and Examples 6A-6E are summarized in Tables 6.1-6.3 below. The solids %-values refer to the solid amounts (Amt) in % by weight.

TABLE 6.1

| | Comp. Ex. 1A | | Comp. Ex. 1B | |
|---|---|---|---|---|
| | Amt. | Solids | Amt. | Solids |
| Ingredient (Trade Name) | [g] | [%] | [g] | [%] |
| Huperzine A | 0.37 | 4.99 | 0.37 | 4.99 |
| Polyacrylate adhesive in ethyl | 6.38 | 85.02 | 6.37 | 84.93 |
| acetate Solids content of 39% | | | | |
| by weight (DURO-TAK ™ 87- | | | | |
| 4287 from Henkel) | | | | |
| Triacetine | 0.75 | 9.99 | — | — |
| Cineol | — | — | 0.76 | 10.08 |
| Total | 7.50 | 100.0 | 7.50 | 100.0 |

| | | |
|---|---|---|
| Area Weight [g/m²] | 96.20 | 88.70 |
| Loading API [μg/cm²] | 480.0 | 442.6 |

TABLE 6.2

| | Ex. 6A | | Ex. 6B | |
|---|---|---|---|---|
| | Amt. | Solids | Amt. | Solids |
| Ingredient (Trade Name) | [g] | [%] | [g] | [%] |
| Huperzine A | 0.37 | 4.96 | 0.38 | 4.98 |
| Polyacrylate adhesive in ethyl | 6.39 | 85.00 | — | — |
| acetate Solids content of 39% | | | | |
| by weight (DURO-TAK ™ 87- | | | | |
| 4287 from Henkel) | | | | |
| Polyacrylate adhesive in ethyl | — | — | 6.38 | 84.62 |
| acetate Solids content of 36.5% | | | | |
| by weight (DURO-TAK ™ 87- | | | | |
| 9301 from Henkel) | | | | |
| Methyl laurate | 0.75 | 10.04 | 0.78 | 10.40 |
| Total | 7.51 | 100.0 | 7.54 | 100.0 |

| | | |
|---|---|---|
| Area Weight [g/m²] | 95.20 | 103.90 |
| Loading API [μg/cm²] | 472.2 | 517.4 |

TABLE 6.3

| | Ex. 6C | | Ex. 6D | | Ex. 6E | |
|---|---|---|---|---|---|---|
| | Amt. | Solids | Amt. | Solids | Amt. | Solids |
| Ingredient (Trade Name) | [g] | [%] | [g] | [%] | [g] | [%] |
| Huperzine A | 0.38 | 5.01 | 0.38 | 5.02 | 0.38 | 5.00 |
| Polyacrylate adhesive in ethyl | 6.38 | 85.04 | 6.42 | 84.67 | 5.61 | 74.84 |
| acetate; Solids content of | | | | | | |
| 39% by weight | | | | | | |
| (DURO-TAK ™ 87-4287 | | | | | | |
| from Henkel) | | | | | | |
| 1-Dodecanol | 0.74 | 9.95 | 0.38 | 5.05 | — | — |
| Methyl laurate | — | — | 0.40 | 5.26 | 0.76 | 10.10 |
| Polyvinylpyrrolidone K90F | — | — | — | — | 0.75 | 10.06 |
| Total | 7.50 | 100.0 | 7.58 | 100.0 | 7.50 | 100.0 |

| | | | |
|---|---|---|---|
| Area Weight [g/m²] | 95.30 | 94.30 | 92.70 |
| Loading API [μg/cm²] | 477.5 | 473.4 | 463.5 |

### Preparation of the Coating Composition

Drug substance, used enhancers and crystallization inhibitors if present, were dispersed in the solvent ethyl acetate. Then the adhesives were added. The resulting mass was stirred for appr. 2 hours at 300 upm.

### Coating of the Coating Composition

The resulting huperzine A-containing coating composition was coated on a polyethylene terephthalate film, which may function as a release liner using for example a film applicator from the company Erichsen according to the solid content of the mixture under consideration of the desired coating dry weight and dried for appr. 15 min at room temperature and appr. 15 min at 60° C. Depending on the target area weight the corresponding film applicator gap is 350 μm.

The coating thickness was chosen such that removal of the solution results in an area weight of the huperzine A-containing layer of approx. 96.20 (Comp. Ex. 1A), 88.70 (Comp. Ex. 1B), 95.20 (Ex. 6A), 103.90 (Ex. 6B), 95.30 (Ex. 6C), 94.30 (Ex. 6D) and 92.70 (Ex. 6E) g/m².

The dried film was then laminated to a backing layer (PET 23 μm) to provide a huperzine A-containing self-adhesive layer structure.

### Preparation of the TTS (Concerning all Examples)

The individual systems (TTS) were then punched out from the huperzine A-containing self-adhesive layer structure obtained as described above. Then, the TTS were sealed into pouches of the primary packaging material.

### Measurement of Skin Permeation

The permeated amount of TTS prepared according to comparative Examples 1A-1B and Examples 6A-E was determined in accordance with the OECD Guideline (adopted Apr. 13, 2004) carried out with a 7.0 mL Franz diffusion cell. Human skin (female) was used. A dermatome was used to prepare skin to a thickness of 500 μm, with an intact epidermis for all TTS. Diecuts with an area of release of 1.164 cm² were punched from the TTS. The huperzine A permeated amount in the receptor medium of the Franz diffusion cell (phosphate buffer pH 5.5 with 0.1% sodium azide as antibacteriological agent) at a temperature of 32±1° C. was measured and the corresponding cumulative permeated amount was calculated.

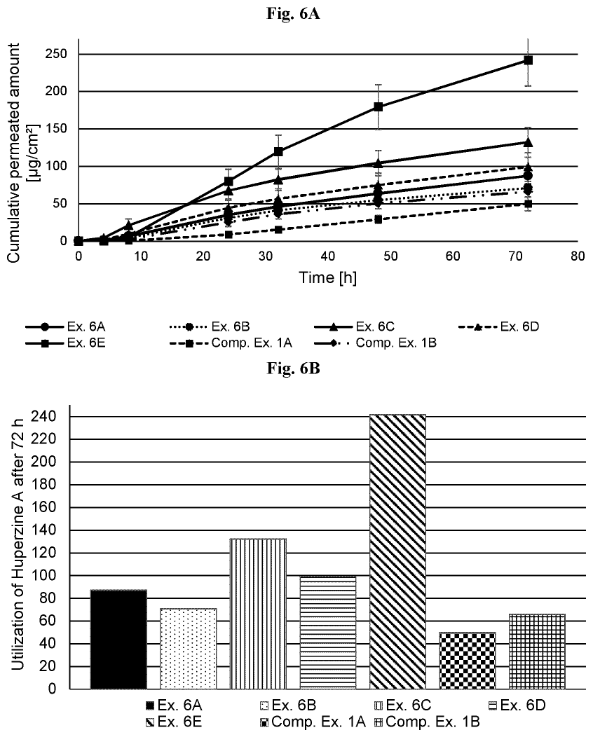

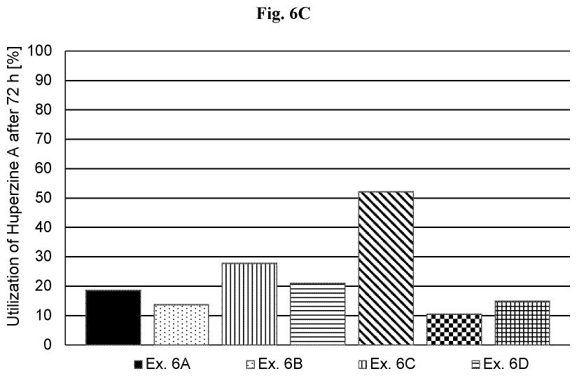

The results are shown in Table 6.4 and 6.5 and FIG. 6A-C.

TABLE 6.4
Cumulative permeated amount with SD [μg/cm²]

| | Comp. Ex. 1A | | Comp. Ex. 1B | | | |
|---|---|---|---|---|---|---|
| Elapsed | (n = 3) | | (n = 3) | | Ex. 6A (n = 3) | |
| time [h] | Amount | SD | Amount | SD | Amount | SD |
| 4 | 0.00 | 0.00 | 0.16 | 0.18 | 0.95 | 0.83 |
| 8 | 0.53 | 0.15 | 2.1 | 1.14 | 6.59 | 3.76 |
| 24 | 8.73 | 1.76 | 25.24 | 5.47 | 35.13 | 6.59 |
| 32 | 15.34 | 3.03 | 35.97 | 6.31 | 46.2 | 7.2 |
| 48 | 29.16 | 5.53 | 50.45 | 6.84 | 63.66 | 8.25 |
| 72 | 49.8 | 9.22 | 65.9 | 6.57 | 87.23 | 10.02 |

TABLE 6.5
Cumulative permeated amount with SD [μg/cm²]

| Elapsed | Ex. 6B (n = 3) | | Ex. 6C (n = 3) | | Ex. 6D (n = 3) | | Ex. 6E (n = 3) | |
|---|---|---|---|---|---|---|---|---|
| time [h] | Amount | SD | Amount | SD | Amount | SD | Amount | SD |
| 4 | 0.38 | 0.24 | 4.12 | 2.77 | 1.27 | 1.43 | 0.1 | 0.09 |
| 8 | 4.06 | 0.99 | 21.05 | 9.00 | 9.71 | 6.22 | 4.98 | 1.66 |
| 24 | 30.96 | 2.3 | 67.46 | 12.98 | 44.6 | 12.09 | 79.94 | 16.16 |
| 32 | 41.09 | 2.64 | 82.12 | 14.31 | 56.51 | 13.63 | 119.48 | 22.39 |
| 48 | 54.64 | 3.37 | 104.23 | 16.96 | 74.92 | 16.02 | 179.25 | 29.89 |
| 72 | 70.85 | 4.87 | 132.16 | 19.81 | 99.16 | 19.08 | 241.53 | 34.09 |

The utilization of huperzine A at 72 h was calculated based on the cumulative permeated amount at 72 h and the initial huperzine A content. The results are shown in Table 6.6 and in FIG. 6A-C.

TABLE 6.6
Utilization of Huperzine A after 72 h [%]

| Comp. | Comp. | | | | | |
|---|---|---|---|---|---|---|
| Ex 1A | Ex 1B | Ex. 6A | Ex. 6B | Ex. 6C | Ex. 6D | Ex. 6E |
| 10.4 | 14.9 | 18.5 | 13.7 | 27.7 | 21.0 | 52.1 |

In one embodiment, the present invention refers to the following preferred items.
1. Transdermal therapeutic system for the transdermal administration of huperzine A comprising a huperzine A-containing layer structure, said huperzine A-containing layer structure comprising:
   A) a backing layer; and
   B) a huperzine A-containing layer;
wherein said huperzine A-containing layer is a huperzine A-containing matrix layer comprising:
   i) huperzine A; and
   ii) at least one polymer selected from the group consisting of silicone polymers, polyisobutylenes, acrylate polymers and styrene-isoprene-styrene block copolymers; and
   iii) at least one additive selected from crystallization inhibitors, permeation enhancers and combinations thereof.
2. The transdermal therapeutic system according to item 1, wherein the at least one polymer is an acrylate polymer or a styrene-isoprene-styrene block copolymer, wherein the acrylate polymer is preferably an acrylic copolymer comprising a hydroxyl group based on vinyl acetate, 2-ethylhexyl-acrylate and 2-hydroxyethyl-acrylate or wherein the polymer is preferably a styrene-isoprene-styrene block copolymer.
3. The transdermal therapeutic system according to item 1 or 2, wherein the at least one polymer is present in an amount of from 65 to 90% by weight, based on the total weight of the huperzine A-containing layer.
4. The transdermal therapeutic system according to any one of items 1 to 3, wherein the crystallization inhibitor is selected from polyvinylpyrrolidone (PVP, povidone), polyvinylpolypyrrolidone (PVPP, crospovidone) and combinations thereof.
5. The transdermal therapeutic system according to any one of items 1 to 4, wherein the crystallization inhibitor is present in an amount of from 5 to 25% by weight, based on the total weight of the huperzine A-containing layer.
6. The transdermal therapeutic system according to any one of items 1 to 5, wherein the permeation enhancer is selected from the group consisting of transcutol, lauryl lactate, methyl laurate, capryol 90, 1-dodecanol, levulinic acid and combinations thereof, and is preferably selected from methyl laurate, capryol 90, 1-dodecanol and combinations thereof.
7. The transdermal therapeutic system according to item 6, wherein the permeation enhancer is methyl laurate.
8. The transdermal therapeutic system according to any one of items 1 to 7, wherein the permeation enhancer is present in an amount of from 2 to 15% by weight, preferably in an amount of from 2.5 to 7.5% by weight, based on the total weight of the huperzine A-containing layer.
9. The transdermal therapeutic system according to any one of items 1 to 8 wherein the transdermal therapeutic system comprises at least two additives, wherein the first additive is a crystallization inhibitor, preferably polyvinylpyrrolidone (PVP, povidone) or polyvinylpolypyrrolidone (PVPP, crospovidone), and the second additive is a permeation enhancer, preferably methyl laurate and/or 1-dodecanol.
10. The transdermal therapeutic system according to any one of items 1 to 9, wherein the huperzine A-containing layer structure comprises huperzine A in an amount of from 1 to 10%, preferably in an amount of from 1.5 to 6% by weight, based on the total weight of the huperzine A-containing layer.
11. The transdermal therapeutic system according to any one of items 1 to 10 wherein the huperzine A-containing layer comprises:
   i) huperzine A; and
   ii) an acrylate polymer, preferably an acrylic copolymer comprising a hydroxyl group based on vinyl acetate, 2-ethylhexyl-acrylate, and 2-hydroxyethyl-acrylate; and
   iii) at least two additives selected from methyl laurate and polyvinylpyrrolidone (PVP, povidone).
12. The transdermal therapeutic system according to any one of items 1 to 10, wherein the huperzine A-containing layer comprises:
   i) huperzine A
   ii) at least one polymer selected from styrene-isoprene-styrene block copolymers; and
   iii) at least two additives selected from methyl laurate and polyvinylpolypyrrolidone (PVPP, crospovidone).
13. The transdermal therapeutic system according to any one of items 1 to 12, wherein the area weight of the huperzine A-containing layer ranges from 60 to 200 g/m², and/or wherein the huperzine A containing layer has a API-loading of 150 to 1200 μg/cm².
14. Transdermal therapeutic system according to any one of items 1-13 for use in a method of treating Alzheimer's disease or any disease related to cognitive dysfunction.
15. Transdermal therapeutic system for use according to item 14, wherein the transdermal therapeutic system is applied to the skin of the patient for at least 24 hours, or 84 hours, or 168 hours.

1. Transdermal therapeutic system for the transdermal administration of huperzine A comprising a huperzine A-containing layer structure, said huperzine A-containing layer structure comprising:
A) a backing layer; and
B) a huperzine A-containing layer;
wherein said huperzine A-containing layer is a huperzine A-containing matrix layer comprising:
i) huperzine A; and
ii) at least one polymer selected from the group consisting of silicone polymers, polyisobutylenes, acrylate polymers and styrene-isoprene-styrene block copolymers; and
iii) at least one additive selected from crystallization inhibitors, permeation enhancers and combinations thereof.

2. The transdermal therapeutic system according to claim 1, wherein the huperzine A-containing matrix layer is a self-adhesive layer structure and wherein the transdermal therapeutic system does not comprise an additional skin contact layer.

3. The transdermal therapeutic system according to claim 1 or 2, wherein the at least one polymer is an acrylate polymer or a styrene-isoprene-styrene block copolymer, wherein the acrylate polymer is preferably an acrylic copolymer comprising a hydroxyl group based on vinyl acetate, 2-ethylhexyl-acrylate and 2-hydroxyethyl-acrylate or wherein the polymer is preferably a styrene-isoprene-styrene block copolymer.

4. The transdermal therapeutic system according to any one of claims 1 to 3, wherein the at least one polymer is present in an amount of from 65 to 90% by weight, based on the total weight of the huperzine A-containing layer.

5. The transdermal therapeutic system according to any one of claims 1 to 4, wherein the crystallization inhibitor is selected from polyvinylpyrrolidone (PVP, povidone), polyvinylpolypyrrolidone (PVPP, crospovidone) and combinations thereof.

6. The transdermal therapeutic system according to any one of claims 1 to 5, wherein the crystallization inhibitor is present in an amount of from 5 to 25% by weight, based on the total weight of the huperzine A-containing layer.

7. The transdermal therapeutic system according to any one of claims 1 to 6, wherein the permeation enhancer is selected from the group consisting of transcutol, lauryl lactate, methyl laurate, capryol 90, 1-dodecanol, levulinic acid and combinations thereof, and is preferably selected from methyl laurate, capryol 90, 1-dodecanol and combinations thereof.

8. The transdermal therapeutic system according to claim 7, wherein the permeation enhancer is methyl laurate.

9. The transdermal therapeutic system according to any one of claims 1 to 8, wherein the permeation enhancer is present in an amount of from 2 to 15% by weight, preferably in an amount of from 2.5 to 7.5% by weight, based on the total weight of the huperzine A-containing layer.

10. The transdermal therapeutic system according to any one of claims 1 to 9 wherein the transdermal therapeutic system comprises at least two additives, wherein the first additive is a crystallization inhibitor, preferably polyvinylpyrrolidone (PVP, povidone) or polyvinylpolypyrrolidone (PVPP, crospovidone), and the second additive is a permeation enhancer, preferably methyl laurate and/or 1-dodecanol.

11. The transdermal therapeutic system according to any one of claims 1 to 10, wherein the huperzine A-containing layer structure comprises huperzine A in an amount of from 1 to 10%, preferably in an amount of from 1.5 to 6% by weight, based on the total weight of the huperzine A-containing layer.

12. The transdermal therapeutic system according to any one of claims 1 to 11 wherein the huperzine A-containing layer comprises:
i) huperzine A; and
ii) an acrylate polymer, preferably an acrylic copolymer comprising a hydroxyl group based on vinyl acetate, 2-ethylhexyl-acrylate, and 2-hydroxyethyl-acrylate; and
iii) at least two additives selected from methyl laurate and polyvinylpyrrolidone (PVP, povidone).

13. The transdermal therapeutic system according to any one of claims 1 to 11, wherein the huperzine A-containing layer comprises:
i) huperzine A
ii) at least one polymer selected from styrene-isoprene-styrene block copolymers; and
iii) at least two additives selected from methyl laurate and polyvinylpolypyrrolidone (PVPP, crospovidone).

14. The transdermal therapeutic system according to any one of claims 1 to 13, wherein the area weight of the huperzine A-containing layer ranges from 60 to 200 g/m², and/or wherein the huperzine A containing layer has a API-loading of 150 to 1200 μg/cm².

15. Transdermal therapeutic system according to any one of claims 1-14 for use in a method of treating Alzheimer's disease or any disease related to cognitive dysfunction.

16. Transdermal therapeutic system for use according to claim 15, wherein the transdermal therapeutic system is applied to the skin of the patient for at least 24 hours, or 84 hours, or 168 hours.
